(19) United States
(12) Patent Application Publication
(10) Pub. No.: US 2026/0240831 A1
(43) Pub. Date: Aug. 20, 2026

(54) CANCER STRATIFICATION AND TREATMENT

(71) Applicant: Deutsches Krebsforschungszentrum Stiftung des öffentlichen Rechts, Heidelberg (DE)

(72) Inventors: Ana MARTIN-VILLALBA, Heidelberg (DE); Oguzhan KAYA, Heidelberg (DE); Leo FÖRSTER, Heidelberg (DE)

(73) Assignee: Deutsches Krebsforschungszentrum Stiftung des öffentlichen Rechts, Heidelberg (DE)

(21) Appl. No.: 19/164,523

(22) PCT Filed: Mar. 15, 2024
(86) PCT No.: PCT/EP2024/056980

§ 371 Date: Sep. 11, 2025

(30) Foreign Application Priority Data
Mar. 15, 2023 (EP) 23162157.4

(51) Int. Cl. A61K 31/4465; A61P 35/00; G01N 33/575

(52) U.S. Cl. CPC A61K 31/4465; A61P 35/00; G01N 33/57557

(57) ABSTRACT

The present invention relates to a modulator of wnt activity (wnt modulator) for use in treating a glioma in a subject, wherein cancer cells of said glioma were allocated to a multitude of lineage subpopulations, and to methods, databases, and uses related thereto.

The present invention relates to a modulator of wnt activity (wnt modulator) for use in treating a glioma in a subject, wherein cancer cells of said glioma were allocated to a multitude of lineage subpopulations, and to methods, databases, and uses related thereto.

Wnt signaling serves as a critical modulator of stem cell self-renewal and differentiation in embryonic development as well as in adult organisms ([44]; [48]; [37]). In multiple tissues, aberrant activation of Wnt has been linked to cancer initiation and/or maintenance, though the underlying molecular mechanisms are not fully studied ([44]; [48]; [37]). Nonetheless, a series of clinical trials related to wnt-signaling in cancer treatment were undertaken (cf. e.g. the review byin [23]).

Glioblastoma multiforme (GBM) remains essentially untreatable due to its infiltrative growth within the vulnerable brain. The heterogeneity and plasticity of the cellular states contribute to poor clinical outcome through treatment resistance and relapse ([39]).

Since the identification of stem-like GBM cells (GSCs) in the adult human brain in the beginning of the 21st century, their counterparts in the healthy brain—namely adult neural stem cells (NSCs) residing in the ventricular sub-ventricular zone (v-SVZ)—have been the prime suspect for tumor cell origin ([36]; [46]; [45]). In more recent years, independent groups experimentally substantiated this hypothesis ([38]; [33]; [34]). Other studies pointed out aberrant re-enforcement of normal neuro-developmental programs in these tumors. In attempts to describe the heterogeneity in GBM, more and more tumor samples have been subjected to omics assays with single cell resolution thanks to the recent technological advancements ([40]; [47]; [35]; [43]).

In GBM, Wnt activity is considered as a driver of tumor replenishment and effective brain colonization ([42]; [41]). The former is shown to be dependent on repression of Dkk1 by the transcription factor Ascl1. On the other hand tumor colonization depends on accumulation of Fzd1 within the tumor microtubes that wreathe around neurons to deplete neuronal Wnt proteins ([35]). Notably, Ascl1 is a crucial pro-activator factor in healthy NSCs, highlighting conserved principles between healthy and malignant NSCs. Also, SFRP1 was found to inhibit glioma growth in vitro ([24]) and wnt pathway regulation was suggested for glioma treatment based on in vitro data ([25], [26]). Despite these lines of experimental evidence for the involvement of Wnt activities in GBM, its role remains unclear.

There is, thus, a need in the art for improved means and methods for treatment of glioma, in particular glioblastoma, and reagents related thereto, avoiding the drawbacks as referred to above. In particular, improved methods for assigning patients to treatments are required, as are new modes of treatment to improve prognosis of glioma.

The technical problem underlying the present invention can be seen as the provision of means and methods for complying with the aforementioned needs. The technical problem is solved by the embodiments characterized in the claims and herein below. Some data related to the invention described herein have been published in [49].

In accordance, the present invention relates to a modulator of wnt activity (wnt modulator) for use in treating a glioma in a subject.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof, thus, e.g. identifying “a cell” relates to identifying at least one cell, preferably to identifying a multitude of cells. The term “multitude” is understood by the skilled person to relate to more than one, i.e. at least two, preferably at least three, more preferably at least four, most preferably at least five. In the context of biomarker evaluation, a multitude may, however, also be a number of at least ten, preferably at least 25, more preferably at least 50, even more preferably at least 100 biomarkers.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin. Preferably, the fragment has the same biological activity as the specifically indicated macromolecule it is derived from, i.e. in particular is a modulator of wnt activity as specified herein below.

Unless specifically indicated otherwise herein, the compounds specified, in particular the polynucleotides and polypeptides, may be comprised in larger structures, e.g. may be covalently or non-covalently linked to further sequences, carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C- or N-terminally to the fusion polypeptide. Said stretch of amino acids preferably allows for detection of the polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences, e.g. an IL-2 signal sequence, and linker sequences.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually considered to be “peptides”. Preferably, the polypeptide comprises of from 50 to 1000, more preferably of from 75 to 1000, still more preferably of from 100 to 500, most preferably of from 110 to 400 amino acids. Preferably, the polypeptide is comprised in a fusion polypeptide and/or a polypeptide complex.

The term “wnt” is known to the skilled person and relates to a large family of structurally related lipid-modified (palmitoylated), secreted signaling glycoproteins that are 350-400 amino acids in length, which are transported to the plasma membrane for secretion and bind to the receptor Frizzled. An exemplary amino acid sequence of a human wnt polypeptide is provided in e.g. Genbank Acc No. NP_005421.1 (human Wnt-1 precursor). The wnt signaling pathways are known to the skilled person as well, e.g. from standard textbooks on cell signaling. In accordance, wnt signaling polypeptides are in particular a wnt polypeptide, LRP5 (Genbank Acc No. NP_001278831.1), LRP6 (Genbank Acc No. AAI43726.1), frizzled (Fzd, Genbank Acc No. AAI43726.1), axin (Genbank Acc No. AAK61224.1), and catenin-beta (Genbanf Acc No. NP_001091679.1). Wnt regulated genes are also known in the art and include in particular SLC7A2 (ENSEMBL gene ID ENSG00000003989); UHRF1 (ENSEMBL gene ID ENSG00000034063); MCM2 (ENSEMBL gene ID ENSG00000073111); TCF7 (ENSEMBL gene ID ENSG00000081059); GRAMD1A (ENSEMBL gene ID ENSG00000089351); TFAP4 (ENSEMBL gene ID ENSG00000090447); FOXRED2 (ENSEMBL gene ID ENSG00000100350); PALD1 (ENSEMBL gene ID ENSG00000107719); RNF43 (ENSEMBL gene ID ENSG00000108375); SLC16A10 (ENSEMBL gene ID ENSG00000112394); LOXL3 (ENSEMBL gene ID ENSG00000115318); CD3EAP (ENSEMBL gene ID ENSG00000117877); ABCC4 (ENSEMBL gene ID ENSG00000125257); BMP4 (ENSEMBL gene ID ENSG00000125378); GINS2 (ENSEMBL gene ID ENSG00000131153); NES (ENSEMBL gene ID ENSG00000132688); GLS2 (ENSEMBL gene ID ENSG00000135423); ADAMTS14 (ENSEMBL gene ID ENSG00000138316); MNS1 (ENSEMBL gene ID ENSG00000138587); LGR5 (ENSEMBL gene ID ENSG00000139292); DTL (ENSEMBL gene ID ENSG00000143476); RFC4 (ENSEMBL gene ID ENSG00000163918); ZNF367 (ENSEMBL gene ID ENSG00000165244); CLDN2 (ENSEMBL gene ID ENSG00000165376); LARGE2 (ENSEMBL gene ID ENSG00000165905); CDT1 (ENSEMBL gene ID ENSG00000167513); SCARA3 (ENSEMBL gene ID ENSG00000168077); AXIN2 (ENSEMBL gene ID ENSG00000168646); CDCA4 (ENSEMBL gene ID ENSG00000170779); CBX2 (ENSEMBL gene ID ENSG00000173894); LRRN1 (ENSEMBL gene ID ENSG00000175928); GINS3 (ENSEMBL gene ID ENSG00000181938); EPHB3 (ENSEMBL gene ID ENSG00000182580); ZNRF3 (ENSEMBL gene ID ENSG00000183579); FAM111B (ENSEMBL gene ID ENSG00000189057); ZNF724 (ENSEMBL gene ID ENSG00000196081); TEAD4 (ENSEMBL gene ID ENSG00000197905); ANKRD13B (ENSEMBL gene ID ENSG00000198720); SP5 (ENSEMBL gene ID ENSG00000204335); FAM216A (ENSEMBL gene ID ENSG00000204856); EMSLR (ENSEMBL gene ID ENSG00000232445).

The term “modulator” is known to the skilled person to relate to any compound causing the activity of a biological molecule or pathway to deviate, preferably significantly, from the activity in the absence of said activity modulator. Preferably, said deviation is a deviation of at least 20%, more preferably at least 50%, even more preferably at least 75%, even more preferably at least 90% of the value of an activity parameter determinable in the absence of said modulator, in particular in case the modulator is an activity decreasing compound; said deviation may, however, also be a deviation by a factor of at least two, preferably at least five, more preferably at least ten, in particular in case the modulator is an activity increasing compound. As is understood by the skilled person in view of the description herein below, modulation may, however, also be complete abolishment of an activity present in the absence of said modulator; or may be de novo activity not present in the absence of the modulator. The effect of the modulator may be temporary, e.g. over a time frame of hours or days, or may be permanent, in particular depending on the specific choice of the modulator. Preferably, said effect is temporary and lasts for of from 1 day to 6 months, preferably of from 2 days to 2 months, more preferably of from 3 days to 4 weeks, most preferably of from 1 to 4 weeks. As will also be understood by the skilled person, the effect may be local, i.e. topical at a site of administration, or may be systemic, e.g. after systemic administration of the modulator.

Preferably, the modulator is an activity decreasing compound, i.e. an inhibitor; thus, preferably the wnt modulator is a wnt signaling decreasing compound, preferably a wnt pathway-specific inhibitor, i.e. preferably is an inhibitor of wnt signaling, i.e. an inhibitor of wnt or of a downstream signaling component. Preferably, the wnt pathway-specific inhibitor inhibits non-wnt-specific activity by at most 50%, preferably at most 25%, more preferably by at most 10% at a concentration inhibiting wnt activity by 90%. The activity of a wnt inhibitor is, preferably, determined in vitro by assaying wnt signaling as specified elsewhere herein, preferably as shown herein in the Examples. Compounds decreasing activity of a known polypeptide gene product such as wnt can be provided by the skilled person by standard methods of molecular biology, e.g. as specified herein below.

Preferably, the wnt inhibitor is a direct wnt inhibitor, i.e. a compound binding to, preferably specifically binding to a wnt signaling component, and thereby inhibiting wnt signaling. More preferably, the direct wnt inhibitor is a small molecule inhibitor, an inhibitor polypeptide, an inhibitor polynucleotide, or a non-polypeptide non-polynucleotide inhibitor macromolecule. Preferably, the direct wnt inhibitor is a compound binding to at least one epitope in a wnt polypeptide or a downstream wnt signaling component, preferably an epitope including at least one amino acid of an interaction epitope and/or an active center of said wnt signaling component. The skilled person is aware of methods suitable for determining binding of a direct inhibitor of wnt signaling, e.g. staining of wnt signaling-positive cells or of extracts from such cells with a candidate direct inhibitor, wherein said inhibitor is coupled to a detectable label, preferably a colored and/or fluorescent dye; ELISA methods; surface plasmon resonance methods, and the like.

The term “small molecule”, as used herein, relates to a chemical molecule with a molecular mas of at most 2.5 kDa, preferably at most 2 kDa, more preferably at most 1.5 kDa, most preferably at most 1 kDa. The “small molecule inhibitor” may, in principle, be from any chemical class of molecules. Preferably, the small molecular inhibitor is an organic molecule, i.e. comprises at least one carbon-carbon bond.

The term “inhibitor polypeptide” is used herein to relate to any and all polypeptides or peptides binding to a wnt polypeptide and/or a downstream component of the wnt pathway and inhibiting its activity. Thus, the inhibitor polypeptide preferably is an antagonist, more preferably a competitive antagonist. Preferably, the inhibitor polypeptide is selected from the list consisting of a compound providing a notum polypeptide (Genbank Acc No. NP_848588.3), a porcupine polypeptide (Genbank Acc No. NP_073736.2), secreted frizzled-related protein 1 (sFRP1, Genbank Acc No. NP_003003.3), or is an inhibitor polypeptide inhibiting dickkopf-related protein 1 (DKK1, Genbank Acc No. NP_036374.1), or a wnt polypeptide. Such an inhibitory polypeptide may in particular be selected from the list consisting of an antibody, an aptamer, an anticalin, and a Designed Ankyrin Repeat Protein (DARPin).

As used herein, the term “antibody” relates to a soluble immunoglobulin from any of the classes IgA, IgD, IgE, IgG, or IgM, or fragments thereof, having the activity of directly interacting with a wnt polypeptide or a downstream wnt signaling polypeptide and inhibiting wnt signaling activity as specified herein above. Antibodies against a wnt signaling component or fragments thereof can be prepared by well-known methods using a purified wnt signaling polypeptide or a suitable fragment derived therefrom as an antigen. A fragment which is suitable as an antigen may be identified by antigenicity determining algorithms well known in the art. Suitable fragments may also be obtained either from a wnt signaling polypeptide by proteolytic digestion, may be synthetic peptides, or may be recombinantly expressed. Suitability of an antibody thus generated as a wnt inhibitor can be tested by an assay as described elsewhere herein. Preferably, the antibody of the present invention is a monoclonal antibody, a human, primatized, chimerized, or humanized antibody, or a fragment thereof. More preferably, the antibody is a single chain antibody, a single-domain antibody, a nanobody, or an antibody fragment, such as Fab, scFab, and the like. Also comprised as antibodies of the present invention are a bispecific antibody, a synthetic antibody, or a chemically modified derivative of any of the aforesaid antibodies. Preferably, the antibody of the present invention shall specifically bind (i.e. does not cross react with other polypeptides or peptides) to a wnt signaling polypeptide as specified above. Specific binding can be tested by various well-known techniques. Antibodies or fragments thereof can be obtained by using textbook methods.

As used herein, the term “aptamer” relates to a polynucleotide or polypeptide binding specifically to a target molecule by virtue of its three-dimensional structure. Preferably, the aptamer is a peptide aptamer, a “peptide aptamer” preferably being a peptide specifically interacting with wnt and, thereby, inhibiting wnt activity as specified herein above. Peptide aptamers, preferably, are peptides comprising 8-80 amino acids, more preferably 10-50 amino acids, and most preferably 15-30 amino acids. They can e.g. be isolated from randomized peptide expression libraries in a suitable host system like baker's yeast (see, for example, [27]).

As used herein, the term “anticalin” relates to an artificial polypeptide derived from a lipocalin specifically binding a wnt signaling polypeptide and inhibiting said polypeptide activity. Similarly, a “Designed Ankyrin Repeat Protein” or “DARPin”, as used herein, is an artificial polypeptide, comprising several ankyrin repeat motifs, specifically binding a wnt signaling polypeptide and inhibiting the activity of said polypeptide. Preferably, the anticalin or DARPin is an anticalin or DARPin as specified above or a polypeptide derivative thereof, more preferably, the anticalin or DARPin is an anticalin or DARPin as specified above.

Also preferably, the wnt inhibitor is a compound not directly interacting with a wnt signaling polypeptide, but still reducing, preferably significantly, wnt signaling activity in a target cell. Preferably, the indirect wnt inhibitor is a compound decreasing the amount of wnt or of a downstream signaling polypeptide in the wnt pathway in a target cell. Preferably, the indirect wnt inhibitor specifically binds to a polynucleotide encoding wnt or a downstream signaling polypeptide, preferably thereby significantly reducing, more preferably preventing, wnt signaling. Also preferably, the indirect wnt inhibitor is or binds to, preferably specifically binds to, a transcriptional regulator of the wnt gene or of a downstream signaling polypeptide, preferably thereby significantly reducing, more preferably preventing, wnt signaling. Thus, the indirect wnt inhibitor may e.g. be a transcriptional repressor of wnt transcription, or may be an inhibitor of a transcriptional activator of wnt transcription. The indirect wnt inhibitor may, however, also be a compound accelerating degradation of a wnt signaling polypeptide in a subject or a compound decreasing the concentration of a wnt activator.

Preferably, the indirect wnt inhibitor is a polynucleotide, more preferably a polynucleotide inhibiting expression or inducing degradation of an mRNA encoding a wnt signaling polypeptide. More preferably, the indirect wnt inhibitor is selected from the group consisting of an shRNA, an siRNA, an miRNA agent, a ribozyme, an antisense molecule/an inhibitory oligonucleotide, and a CRISPR/Cas oligonucleotide. It is understood by the skilled person that inhibition of expression or induction of degradation of a specific RNA can be achieved in various ways. It is also understood by the skilled person that the exact embodiment of a polynucleotide being an indirect wnt inhibitor of the present invention will depend on the treatment intended.

Preferably, the indirect wnt inhibitor is a ribozyme. The term “ribozyme” as used herein, refers to catalytic RNA molecules possessing a well-defined tertiary structure that allows for catalyzing either the hydrolysis of one of their own phosphodiester bonds (self-cleaving ribozymes), or the hydrolysis of bonds in other RNAs, but they have also been found to catalyze the aminotransferase activity of the ribosome. The ribozymes envisaged in accordance with the present invention are, preferably, those which specifically hydrolyze their target RNAs, preferably an mRNA encoding a wnt signaling polypeptide, i.e., e.g. preferably RNA transcribed from a wnt gene. In particular, hammerhead ribozymes are preferred in accordance with the present invention. How to generate and use such ribozymes is well known in the art (see, e.g., Hean & Weinberg (2008), RNA and the Regulation of Gene Expression: A Hidden Layer of Complexity, Chapter 1. Caister Academic Press).

More preferably, the indirect wnt inhibitor is an antisense oligonucleotide. The term “antisense oligonucleotide” is known to the skilled person and relates to an oligonucleotide hybridizing to a target RNA, causing the formation of a DNA/RNA hybrid. Said DNA/RNA hybrid is a substrate for RNase H, which degrades the RNA portion of said DNA/RNA hybrid. Preferably, the antisense oligonucleotide has a length of at least 15 nucleotides, preferably at least 18 nucleotides, still more preferably at least 20 nucleotides, preferably complementary to an mRNA sequence encoding a wnt signaling polypeptide as specified herein above.

Most preferably, the indirect wnt inhibitor is a polynucleotide inducing RNA interference. As used herein, “RNA interference (RNAi)” refers to sequence-specific, post-transcriptional gene silencing of a selected target gene by degradation of RNA transcribed from the target gene (target RNA). The target RNA, preferably, is an mRNAs encoding a wnt signaling polypeptide. RNAi requires in the target cell the presence of dsRNAs that are homologous in sequence to the target RNAs. The term “dsRNA” refers to RNA having a duplex structure comprising two complementary and anti-parallel nucleic acid strands. The RNA strands forming the dsRNA may have the same or a different number of nucleotides, whereby one of the strands of the dsRNA can be the target RNA. It is, however, also contemplated by the present invention that the dsRNA is formed between two sequence stretches on the same RNA molecule, e.g. by formation of a stem-loop structure. RNAi may be used to specifically inhibit expression of the target RNAs of the present invention in vivo. Methods relating to the use of RNAi to silence genes in animals, including mammals, are known in the art.

Thus, the indirect wnt inhibitor preferably is an RNAi agent. As used herein, the term “RNAi agent” refers to an shRNA, a siRNA agent, or a miRNA agent as specified below. The RNAi agent of the present invention is of sufficient length and complementarity to stably interact with the target RNA, i.e. it comprises at least 15, at least 17, at least 19, at least 21, at least 22 nucleotides complementary to the target RNA. By “stably interact” is meant interaction of the RNAi agent or its products produced by the target cell with a target RNA, e.g., by forming hydrogen bonds with complementary nucleotides in the target RNA under physiological conditions.

The term “siRNA agent” as meant herein encompasses: a) a dsRNA consisting of at least 15, at least 17, at least 19, at least 21 consecutive nucleotides base-paired, i.e. forming hydrogen bonds with complementary nucleotides. b) a small interfering RNA (siRNA) molecule or a molecule comprising an siRNA molecule. The siRNA is a single-stranded RNA molecule with a length, preferably, greater than or equal to 15 nucleotides and, preferably, a length of 15 to 49 nucleotides, more preferably 17 to 30 nucleotides, and most preferably 17 to 30 nucleotides, preferably 17, 18, 19, 20, 21, 22, 23, 24, 25, 26, 27, 28, 29, or 30 nucleotides. According to the present invention, the term “molecule comprising an siRNA molecule” includes RNA molecules from which an siRNA is processed by a cell, preferably by a mammalian cell. Thus, a molecule comprising an siRNA molecule, preferably, is a small hairpin RNA, also known as shRNA. As used herein, the term “shRNA” relates to a, preferably artificial, RNA molecule forming a stem-loop structure comprising at least 10, preferably at least 15, more preferably at least 17, most preferably at least 20 nucleotides base-paired to a complementary sequence on the same mRNA molecule (“stem”), i.e. as a dsRNA, separated by a stretch of non-base-paired nucleotides (“loop”). c) a polynucleotide encoding a) or b), wherein, preferably, said polynucleotide is operatively linked to an expression control sequence. Thus, the function of the siRNA agent to inhibit expression of the target gene can be modulated by said expression control sequence. Preferred expression control sequences are those, which can be regulated by exogenous stimuli, e.g. the tet operator, whose activity can be regulated by tetracycline, or heat inducible promoters. Alternatively or in addition, one or more expression control sequences can be used which allow tissue-specific expression of the siRNA agent.

It is, however, also contemplated by the current invention that the RNAi agent is a miRNA agent. A “miRNA agent” as meant herein encompasses: a) a pre-microRNA, i.e. an mRNA comprising at least 30, at least 40, at least 50, at least 60, at least 70 nucleotides base-paired to a complementary sequence on the same mRNA molecule (“stem”), i.e. as a dsRNA, separated by a stretch of non-base-paired nucleotides (“loop”). b) a pre-microRNA, i.e. a dsRNA molecule comprising a stretch of at least 19, at least 20, at least 21, at least 22, at least 23, at least 24, at least 25 base-paired nucleotides formed by nucleotides of the same RNA molecule (stem), separated by a loop. c) a microRNA (miRNA), i.e. a dsRNA comprising at least 15, at least 17, at least 18, at least 19, at least 21 nucleotides on two separate RNA strands. d) a polynucleotide encoding a) or b), wherein, preferably, said polynucleotide is operatively linked to an expression control sequence as specified above.

Also preferably, the indirect wnt inhibitor comprises at least one, preferably two, CRISPR/Cas oligonucleotides. The CRISPR/Cas system has been known for several years as a convenient system for inducing knock-out mutations, i.e. deletions, preferably of chromosomal genes. The skilled person knows how to design appropriate oligonucleotides, which are, preferably, expressed from a vector, to induce deletion of a DNA sequence of interest. Preferably, said deletion is a partial deletion, more preferably deletion of a portion of the gene essential for function; most preferably said deletion is a complete deletion of at least the whole coding region. As is known in the art, single CRISPR/Cas oligonucleotides can be used to introduce short insertions, deletions, and/or frameshifts in a coding sequence of interest, while two CRISPR/Cas oligonucleotides may be used to mediate larger deletions or deletions of exons, coding regions and/or whole genes.

Also preferably, the indirect wnt inhibitor is a polypeptide comprising a lysosome-degradation sequence, preferably a chaperone-mediated autophagy-targeting motif (CTM). Preferably, said CTM-comprising polypeptide specifically binds to a wnt or a downstream signaling polypeptide; e.g. the CTM-comprising polypeptide may further comprise an antibody specifically binding to wnt. As the skilled person will understand, the CTM-conjugated antibody does not necessarily have to be an inhibitory antibody as specified herein above; it is, however, preferred that the antibody is an antibody specific for a wnt signaling polypeptide. As will be understood by the skilled person, in case the indirect wnt inhibitor is a CTM-comprising polypeptide, said CTM-comprising polypeptide does not have to be, but may be, a direct wnt inhibitor. Thus, preferably, the CTM-comprising polypeptide also is a direct wnt inhibitor.

Also preferably, the modulator is an activity increasing compound; thus, preferably the wnt modulator is a wnt activator, preferably a wnt pathway-specific activator. Preferably, the wnt-specific activity increasing compound activates non-wnt-specific activity by at most 50%, preferably at most 25%, more preferably by at most 10%, at a concentration activating wnt activity by 90%. The activity of a wnt activator is, preferably, determined in vitro by assaying the activity of wnt signaling by methods known in the art. Compounds increasing activity of a known polypeptide gene product such as wnt signaling polypeptides can be provided by the skilled person by standard methods of molecular biology, e.g. as specified herein below.

Preferred wnt activators are inhibitors of sFRP1, preferably WAY-316606 (5-(phenylsulfonyl)-N-4-piperidinyl-2-(trifluoromethyl)-benzenesulfonamide, CAS No. 915759-45-4). Also preferred wnt activators are notum inhibitors, preferably small molecule notum inhibitors, reviewed e.g. in [28], in particular LP-935001 (6-chloro-8-fluoro-4,5-dihydrobenzo[g][1]benzothiole-2-carboxylic acid), LP-922056 (2-((6-Chloro-7-cyclopropylthieno[3,2-d]pyrimidin-4-yl)thio)acetic acid, CAS No. 1365060-22-5), LP-914822, or ABC99 (7-(4-Chlorobenzyl)-1,3-dioxohexahydroimidazo[1,5-a]pyrazin-2(3H)-yl 2,3-dihydro-4Hbenzo[b][1,4]oxazine-4-carboxylate).

The term “compound providing polypeptide X”, as referred to herein, relates to any composition of matter causing a polypeptide X to become present, in particular after its application to a cell, preferably to a subject. Thus, the agent providing polypeptide X may be any peptide or polypeptide comprising polypeptide X. From such an agent providing polypeptide X, polypeptide X may be liberated, e.g. by proteolysis (e.g. by a proteasome), by hydrolysis, e.g. of an amido or ester bond to a carrier molecule, and/or by fusion of a lipid vesicle, e.g. of a nanoemulsion, with a cell membrane. Corresponding compositions and methods are known in the art. Also preferably, the agent providing polypeptide X is a nanoemulsion comprising at least polypeptide X; corresponding compositions are known in the art. Also preferably, the agent providing polypeptide X may be the polypeptide X as such.

The agent providing polypeptide X may, however, also be an agent causing a host cell to synthesize polypeptide X or a polypeptide comprising the same; for the peptides and polypeptides which may be produced from such an agent, reference is made to the description herein above. A corresponding agent providing a polypeptide X may in particular be a polynucleotide encoding at least polypeptide X or a polypeptide comprising the same, preferably a polynucleotide encoding at least polypeptide X. The polynucleotide may be any polynucleotide deemed appropriate by the skilled person for the intended use, taking into account e.g. mode of administration, target cell, required dose and duration of expression, and the like. Thus, the agent providing polypeptide X may be an mRNA, an expression construct, optionally comprised in a vector, and the like. Thus, the agent providing polypeptide X preferably is an mRNA or a DNA, preferably double-stranded DNA.

Preferably, the wnt activator is a direct wnt activity increasing compound binding to and activating activity of a wnt signaling polypeptide, more preferably is a small molecule activator, an activator polypeptide, an activator polynucleotide, or a non-polypeptide non-polynucleotide activator macromolecule.

The term “small molecule” has been specified herein above. In accordance, the term “small molecule activator”, as used herein, relates to a small molecule compound increasing wnt activity. Small molecule activators of wnt are known in the art, such as WAY-316606.

The term “activator polypeptide”, as used herein, includes any and all polypeptides having an activating effect on wnt signaling. Preferably, the activator polypeptide is an activator antibody, preferably a wnt signaling agonist antibody, or is a wnt signaling polypeptide or a fragment thereof having the activity of being a wnt activator. Preferably, the activator polypeptide is a wnt polypeptide or a downstream wnt signaling polypeptide.

Also preferably, the wnt activator is an indirect wnt activity increasing compound, i.e. a compound not binding to and not being a wnt signaling polypeptide but nonetheless increasing wnt signaling activity. Preferably, the indirect wnt activator is (i) a polynucleotide encoding a wnt polypeptide or a downstream wnt signaling polypeptide; (ii) a vector comprising the polynucleotide of (i); (iii) a host cell comprising the polynucleotide of (i) and/or the vector of (ii); or (iv) any combination of (i) to (iii).

The term “subject”, as referred to herein, relates to a vertebrate animal, preferably a mammal, in particular a livestock, companion, or laboratory animal. Most preferably, subject is a human. Preferably, the subject has been diagnosed to suffer from a glioma or is suspected to suffer from a glioma. Preferably, the glioma of said subject has been identified to be susceptible to treatment with a wnt modulator, preferably as specified herein below, and/or the glioma of said subject has been stratified as described herein elsewhere.

The term “cancer”, as used herein, relates to a disease of a subject, characterized by uncontrolled growth by a group of body cells (“cancer cells”). This uncontrolled growth may lead to tumor formation and may be accompanied by intrusion into and destruction of surrounding tissue (invasion) and possibly spread of cancer cells to other locations in the body (metastasis). Thus, the cancer preferably is a solid cancer, a metastasis, and/or a relapse thereof. Preferably, the cancer is a neurological cancer, preferably a brain cancer, more preferably a glioma. Symptoms and diagnostic methods for diagnosing the aforesaid cancers are known from standard medical textbooks. Preferably, the glioma is an astrocytoma, preferably a glioblastoma, more preferably a glioblastoma multiforme.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50%, at least 60%, at least 70%, at least 80%, or at least 90% of the subjects of a given cohort or population. Preferably, treating cancer is reducing tumor burden and/or cancer cell load in a subject. As will be understood by the skilled person, methods and effectiveness of treatment of e.g. cancer is dependent on a variety of factors including, e.g. cancer stage and cancer type. In view of the description provided herein, treating may additionally comprise, e.g. may be preceded, accompanied, and/or followed, by surgery, chemotherapy, radiotherapy, targeted therapy, and/or immunotherapy.

The treatment of glioma as specified herein preferably is adapted based on an allocation of cells of said glioma to a multitude of lineage subpopulations, wherein said allocation preferably is based on a lineage analysis of non-cancer cells of the astrocyte lineage. In such lineage analysis, at least three lineage biomarkers is provided; appropriate lineage biomarkers e.g. for the non-cancer astrocyte lineage are known in the art. Said lineage marker may be used to establish a multitude of subpopulations, e.g. two, preferably three, subpopulations, representing different pseudotime values and/or different states of said cells, e.g. activation states. Thus, preferably, e.g. non-cancer cell lineage subpopulations corresponding to quiescent cells (quiescent subpopulation), activated cells (activation subpopulation), and differentiating cells (differentiation subpopulation) may be defined, e.g. based on a set of biomarkers of non-cancer cells. Thus, e.g. activation and/or differentiation biomarkers may be used.

Using determination of the same lineage biomarkers or a subset thereof, cancer cells, e.g. from a glioma, are allocated to subgroups corresponding to the aforesaid lineage subgroups, which may also be referred to a “pseudolineage” subgroups in case they comprise cancer cells. In a preferred embodiment, only data from non-cycling (i.e. non-dividing) cells are taken into account in the aforesaid allocation; in a further preferred embodiment, cycling cells are identified by the same criteria as are used for providing a fixed lineage reference, as described herein below and in the Examples.

Preferred biomarkers for a non-cancer astrocyte lineage are described herein below in Table 1. As is understood by the skilled person from the description herein, in particular the Examples, allocation of cancer cells may lead to a percent distribution of cancer cells over the pre-defined subgroups; thus, selection of treatment modes for a given glioma preferably depends on the outcome of such allocation. Preferably, in case a large fraction of cells is found to be non-quiescent, i.e. is e.g. in the activation or differentiation subpopulation, it may be preferable to treat said cancer with an agent inducing quiescence in order to slow or prevent tumor growth. On the other hand, if a large proportion of cells of a cancer is allocated to a quiescent subpopulation, it may be envisaged to treat said cancer with an activating compound inducing activation, in order to make such cells sensitive to radiotherapy and/or chemotherapy.

In case of glioma, in particular glioblastoma, treatment, e.g. three subpopulations may be pre-defined, e.g. a quiescent subpopulation, an activation subpopulation, and differentiation subpopulation. In case a large fraction, preferably at least 25%, preferably at least 35%, more preferably at least 50%, of tumor cells of said glioma are from an activation subpopulation or a differentiation subpopulation, the treatment preferably comprises administration of a wnt inhibitor. Such treatment may be in particular envisaged in cases in which no chemotherapy is planned for the subject; or in case the treatment is conservative or palliative treatment, e.g. in a subject of at least 50 years, preferably at least 60 years, more preferably at least 70 years, most preferably at least 80 years, of age, in whom prognosis may be unfavorable. Also, administration of a wnt inhibitor may be envisaged after glioma diagnosis and preferably up to surgery, e.g. to reduce or prevent tumor growth until surgery. In case radiotherapy, e.g. intraoperative radiotherapy, and/or chemotherapy, e.g. intraoperative implantation of a long-term chemotherapy pellet at the site of tumor excision are planned, the treatment preferably comprises administration of said wnt inhibitor until surgical resection of said glioma or a part thereof, preferably until at most 12h, preferably at most 1d, more preferably at most 2d, before surgical resection of said glioma of a part thereof.

Preferably, the aforesaid three lineage subpopulations comprise a quiescent subpopulation, an activation subpopulation, and a differentiation subpopulation, and the wnt modulator is for treatment of glioma. In such case, the lineage biomarker is preferably selected from Table 1. Preferably, at least 3, more preferably at least five, even more preferably at least ten, still more preferably at least 25, still more preferably at least 50, even more preferably at least 100 lineage biomarkers are selected independently from the lineage biomarkers of Table 1 herein below. More preferably, at least one of said lineage biomarkers is a quiescence biomarker, at least one of said lineage biomarkers is an activation biomarker, and at least one of said lineage biomarkers is a differentiation biomarker. Also preferably, at least 3, more preferably at least five, even more preferably at least ten, still more preferably at least 25, still more preferably at least 50, even more preferably at least 100 lineage biomarkers are selected and at least 20% of said biomarkers are quiescence biomarkers, at least 20% of said lineage biomarkers are activation biomarkers, and/or at least 20% of said lineage biomarkers are differentiation biomarkers. Most preferably, all lineage biomarkers of Table 1 are determined. It is, however, also envisaged that lineage biomarkers for only two subpopulations according to Table 1 are determined, e.g. only quiescence biomarkers and activation biomarkers, if only a differentiation between these two subpopulations is desired. The lineage biomarkers of Table 1 are indicative for a specific subpopulation in case a gene indicated in Table 1 under said subpopulation is overexpressed, preferably compared to the average of all cells analyzed, i.e. preferably, for all genes of Table 1 at least 60% of the mean counts of that gene across subpopulations stem from the subpopulation it is assigned to. It will be appreciated that Genbank Acc Nos. in Table 1 relate to protein sequences. As referred to herein, the lineage biomarker may be any molecule comprising the indicated sequence or encoding the indicated sequence; i.e. the lineage biomarker may be determined as a polypeptide or fragment thereof, e.g. by immunologic means, or as a polynucleotide, e.g. mRNA or a fragment thereof, more preferably, a plurality of biomarkers is determined by single cell sequencing of expressed genes; in such case, preferably all transcript isoforms belonging to a gene are collapsed during mapping and counted equally.

In case a large fraction, preferably at least 25%, preferably at least 35%, more preferably at least 50%, of tumor cells of said glioma are from an activation subpopulation, it may be envisaged to administer radiotherapy and/or chemotherapy, in order to kill actively dividing cancer cells. However, it may also be envisaged to administer compounds inducing differentiation.

In case a large fraction, preferably at least 25%, preferably at least 35%, more preferably at least 50%, of tumor cells of said glioma are from an activation subpopulation or a differentiation subpopulation, also a biphasic treatment may be envisaged, preferably comprising administration of a wnt inhibitor in a first step, e.g. after diagnosis and preferably until surgery, followed by a second step of administration of a wnt activator in combination with radiotherapy and/or chemotherapy. As the skilled person understands in view of the description herein elsewhere, in the first phase glioma cells are driven to quiescence and stop growing, while the second phase induces activation, preferably essentially synchronized activation, of glioma cells, making said cells sensitive to radiotherapy and/or chemotherapy. Preferably, said first and second phase are repeated, in order to optimize the number of glioma cells actively dividing during chemotherapy and/or radiotherapy.

The terms “radiation therapy” and “radiotherapy” are known to the skilled artisan. The terms relate to the use of ionizing radiation to treat or control cancer. The skilled person also knows the term “surgery”, relating to invasive measures for treating cancer, in particular excision of tumor tissue.

As used herein, the term “chemotherapy” relates to treatment of a subject with an antineoplastic drug. Preferably, chemotherapy is a treatment including alkylating agents (e.g. cyclophosphamide), platinum (e.g. carboplatin), anthracyclines (e.g. doxorubicin, epirubicin, idarubicin, or daunorubicin) and topoisomerase II inhibitors (e.g. etoposide, irinotecan, topotecan, camptothecin, or VP16), anaplastic lymphoma kinase (ALK)-inhibitors (e.g. Crizotinib or AP26130), aurora kinase inhibitors (e.g. N-[4-[4-(4-Methylpiperazin-1-yl)-6-[(5-methyl-1H-pyrazol-3-yl)amino]pyrimidin-2-yl]sulfanylphenyl]cyclopropanecarboxamide (VX-680)), antiangiogenic agents (e.g. Bevacizumab), or Iodine131-1-(3-iodobenzyl)guanidine (therapeutic metaiodobenzylguanidine), alone or any suitable combination thereof. It is to be understood that chemotherapy, preferably, relates to a complete cycle of treatment, i.e. a series of several (e.g. four, six, or eight) doses of antineoplastic drug or drugs applied to a subject separated by several days or weeks without such application. As the skilled person understands, chemotherapy of glioma may in particular be coordinated with surgery, e.g. by starting chemotherapy after or during surgery, e.g. by implanting a long-term chemotherapy pellet at a site of tumor excision.

The term “targeted therapy”, as used herein, relates to application to a patient of a chemical substance known to block growth of cancer cells by interfering with specific molecules known to be necessary for tumorigenesis or cancer or cancer cell growth. Examples known to the skilled artisan are small molecules like, e.g. PARP-inhibitors (e.g. Iniparib), or monoclonal antibodies like, e.g., Trastuzumab. The term “immunotherapy” as used herein relates to the treatment of cancer by modulation of the immune response of a subject, e.g. as cell based immunotherapy. Said modulation may be inducing, enhancing, or suppressing said immune response. The term “cell based immunotherapy” relates to a cancer therapy comprising application of immune cells, e.g. T-cells, preferably tumor-specific NK cells, to a subject.

Advantageously, it was found in the work underlying the present invention that cancer cell populations can be allocated to subpopulations normally relevant only for lineage of development of normal, i.e. non-cancer cells. Even more surprisingly, it was found that allocation to lineage subgroups predicts physiological properties of the cancer and makes predictions on cancer sensitivity to specific treatments and cancer prognosis possible.

The definitions made above apply mutatis mutandis to the following. Additional definitions and explanations made further below also apply for all embodiments described in this specification mutatis mutandis.

The present invention further relates to a use of a wnt modulator for the manufacture of a medicament for treating glioma, preferably as specified herein above.

The present invention also relates to a method for determining whether a subject suffering from a glioma is susceptible to treatment with a wnt modulator, said method comprising
(a) determining at least three lineage biomarkers in a sample of cancer cells of said subject,
(b) comparing said at least three lineage biomarkers determined in step (a) to a lineage reference; and
(c) identifying a subject susceptible to treatment with a wnt modulator based on the result of comparing step (b).

The method of determining, preferably, is an in vitro method. Moreover, it may comprise steps in addition to those explicitly mentioned above. For example, further steps may relate, e.g., to pre-determining at least one suitable lineage biomarker for step a), or further assessments in addition to those of steps (a) and (b), such as cancer staging. Moreover, one or more of said steps may be assisted or performed by automated equipment.

As used herein, the term “susceptible to treatment” relates to the property of a subject and/or a cancer thereof, i.e. of cells of said cancer, to be inhibited, killed, and/or prevented from migration and/or invasion into healthy tissue by said treatment. Thus, in a subject susceptible to treatment as described herein, upon administration of an effective dose of a wnt modulator and optionally further treatment as described herein above, e.g. radio- and/or chemotherapy, said cancer preferably stops growing, more preferably is reduced in mass (partial remission), most preferably is completely removed from the subject (complete remission). Thus, preferably, in a subject susceptible to treatment cancer cells are inhibited from growing, more preferably at least partially lyse upon said treatment. As the skilled person understands from the description herein above, the subject may be found to be susceptible to treatment with one therapy, e.g. treatment with a wnt inhibitor, or a combination therapy with a wnt activator with radio- and/or chemotherapy, but may also be found to be susceptible to a multi-phase treatment, e.g. by first treating with a wnt inhibitor, followed by a combination therapy with a wnt activator with radio- and/or chemotherapy, or vice versa. Using the method of determining, optionally repeatedly, treatment steps can be optimized in accordance with the physiological state of the cancer cells. It will also be understood that the aforesaid method of determining is preferably independent from mutation status staging markers used in the art for staging cancer cells.

The term “lineage biomarker” is in principle understood by the skilled person. Preferably, the term includes each and every measurable feature of a non-cancer cell which is indicative of said cell's development status. As the skilled person understands, lineage biomarkers are known in the art and depend on the specific lineage the cells under investigation are from. Well-known lineages are e.g. the astrocyte lineage, hematopoietic cell lineage, the epidermal cell lineage, muscle cell lineage, and the like. As referred to herein, a cell lineage preferably starts with a tissue-specific stem cells, e.g. a glial stem cell, and ends with a differentiated cell, e.g. an astrocyte. Lineage biomarkers for cell lineages are known in the art. However, as the term is referred to herein, the lineage biomarker may also be a hitherto unknown biomarker; also, as referred to herein, a lineage biomarker may also be identified in a non-identical animal, preferably from the same family as the subject. Thus, a lineage biomarker for a human cancer may be identified in a laboratory animal, preferably a mammalian animal, more preferably a rat or a pig, more preferably in a mouse. Methods for identifying lineage markers are known in the art and include in particular allocating cells of a lineage to a pre-defined set of subpopulations, of which each may e.g. represent a specific development state; within these groups, preferably a number of candidate lineage biomarkers is determined and evaluated for the capacity to differentiate between the pre-defined subgroups. Appropriate methods, in particular statistical methods, are known in the art. From the biomarkers enabling differentiation between at least two of said pre-defined subgroups, preferably those enabling most reliable differentiation, one or more biomarkers may be selected as lineage biomarkers of the present invention. As referred to herein, the lineage biomarker preferably is a single cell lineage biomarker, i.e. preferably is a biomarker determinable in or on a single cell enabling allocation of said cell to a lineage subgroup. Also preferably, the lineage biomarker is a marker detectable in at least a fraction of cancer cells from the same lineage at levels above the detection limit. Preferably, a lineage biomarker is expression of at least one gene selected from Table 1. Also preferably, the lineage biomarker is a methylation status of at least one gene. In a preferred embodiment, the lineage biomarker is draxin (Genbank Acc No. XP_054192416.1); in another preferred embodiment, the lineage biomarker is not draxin.

As described herein below, in a preferred embodiment, a lineage biomarker may also be determined by bulk determination of said lineage biomarker, wherein the term “bulk determination” relates to a determination of the lineage biomarker in a plurality of cells, e.g. in at least two, in a further preferred embodiment at least three, in a further preferred embodiment at least ten, in a further preferred embodiment at least 100, in a further preferred embodiment at least 1000 cells. Thus, bulk determination may e.g. comprise determination in a biopsy sample or an aliquot thereof. As also detailed herein below, in bulk determination, the same biomarkers as in single-cell determination may be used; however, also a set of bulk lineage biomarkers may be used, in particular those of Table 2. In accordance, in a preferred embodiment, a lineage biomarker is expression of at least one gene selected from Table 2. Also in a preferred embodiment, the lineage biomarker is a methylation status of at least one gene encoding a polypeptide shown in Table 2. Moreover, it will be appreciated that some lineage biomarkers are biomarkers of Table 1 and of Table 2. These lineage biomarkers may in a preferred embodiment also be referred to as “universal” lineage biomarkers, which in a preferred embodiment may be used in single-cell and bulk analysis; thus, the aforesaid universal lineage biomarkers in a preferred embodiment are ALDH1L1, ALDOC, AQP4, ATP1B2, BAALC, CA2, CLU, CXCL14, DHRS3, DKK3, EDNRB, F3, FAM107A, HEPACAM, HOPX, HTRA1, IL33, LIMCH1, MGST1, MLC1, MT3, NTM, PBXIP1, RAMP1, SDC4, SFXN5, SLC4A4, SPARC, SPARCL1, TMEM176A, TRIL, TTYH1, and/or VCAM1 as biomarkers of a quiescent state, ANP32B, BTG2, DLL1, EGR1, FOSB, HELLS, JUNB, KLF4, LIMA1, LYAR, MCM2, MCM3, MCM5, MCM6, NR4A1, OLIG2, PCNA, PPP1R15A, SLBP, TIPIN, TMEM132B, UHRF1, and/or UNG as biomarkers of an activation state, and/or ANKS1B, BASP1, CDK5R1, CELF4, DBN1, DCX, DLX2, DPYSL3, ELAVL3, ELAVL4, FNBP1L, GAD1, GNG2, KIF5C, MAP1B, MEX3A, MLLT11, MPPED2, MYT1L, NOL4, NREP, PAFAH1B3, PLXNA4, SHTN1, STMN2, STMN4, TTC9B, UCHL1, and/or ZNF704 as biomarkers of a differentiation state. In a further preferred embodiment, the lineage biomarker, in particular the universal lineage biomarker, is not ACSS3, ADCYAP1R1, AGT, AHNAK, ARAP2, ATP1A2, BBOX1, BDH2, C21orf62, C3, CCDC80, CHI3L1, CRB2, CRYAB, CSF1, EFEMP1, EFHD1, ENKUR, FADS2, FAM181A, FGF1, GFAP, GLIS3, GPR37, HIF3A, HNMT, HRH1, HSPB8, ID3, ID4, ITGA6, ITM2C, ITPKB, KCNN3, LAMB2, LFNG, LIFR, LRIG1, MAOB, NDP, NDRG2, NMB, NTRK2, PIFO, PLA2G5, PLCD3, PLTP, PON2, PROS1, RFX4, RGMA, RHPN1, ROM1, SCARA3, SLC1A2, SLC1A3, SLC25A18, SMOX, SPOCD1, SSPN, TGFB2, TIMP3, TIMP4, TMEM47, TRIM47, TTYH2, VAMP5, ALKBH2, APOD, ARC, ARL4A, ATF3, BARD1, BEST3, C11orf24, CDCA7L, CENPH, CENPK, CENPU, CENPW, CKS2, CLSPN, CSRNP1, DBF4, DUSP6, EGR2, ERF, EXOSC9, FBL, FEN1, GINS2, GPATCH4, IFRD2, ITGB3BP, JAG1, KLF2, MAFF, MCM4, METTL1, MTHFD2, MYADM, MYC, NEU4, NFKBIZ, PDGFRA, PDLIM1, PPIH, PTBP1, PYCR1, RGS16, RPA2, SERTAD1, TRIB1, TYMS, WEE1, ACTL6B, ADD2, AFAP1, ANK3, ARL4D, ASPHD1, ATL1, B4GALNT1, BCL11A, BEND5, CCNG2, CCSAP, CDC42EP3, CELF3, CEP170, CERS6, CHGB, CSRNP3, CXADR, D name, DIRAS1, DPYSL5, DUSP26, DYNC1I1, ELAVL2, GDAP1, GDAP1L1, GNG3, GPR161, HRK, JPH4, KALRN, KIAA1549, KIF5A, KLF12, KLHDC8A, MAPK10, MTURN, NOVA2, PARP6, PDZD4, PKIA, POU2F2, PRKCZ, PROX1, RAB3A, RBFOX2, REEP1, RGMB, RNF165, ROBO2, RPS6KL1, SBK1, SCN3A, SCN3B, SEZ6, SEZ6L, SEZ6L2, SH3BP5, SNAP25, SOBP, SRRM3, STXBP1, SYT1, THSD7A, TMEM178B, TRIM36, TUBB4A, WDR47, ZBTB8A, ZNF711, or ZNF821.

For one or more lineage biomarker(s), a biomarker reference may be determined. The term “reference”, as used herein, relates to a value, e.g. an amount or any value derived therefrom, e.g. a score, which can be correlated to a lineage subgroup and, preferably, which allows for the assessment of the invention to be made, in a further embodiment enables allocation of a cell to a lineage subgroup. Such a reference can be a threshold value, e.g. a threshold amount, which separates these groups from each other. Accordingly, the reference may be a value which allows for allocation of a cell into a group of cells belonging to a lineage subgroup, or not. For example, the reference may be a value which allows for allocation of a cell into a group of cells being in a quiescent state, an activated state, or a differentiated state. The reference may, however, also be a reference range. Furthermore, the reference may be a value calculated from the aforesaid values, e.g. from the amounts of two or more biomarkers, preferably to provide a score. A suitable reference separating the subgroups can be provided without further ado e.g. by the statistical tests referred to herein elsewhere based on values of biomarkers from suitable reference subgroups as specified herein elsewhere. As the skilled person understands, it may not always be possible, although preferred, to provide a lineage reference unambiguously allocating each and every possible value of a lineage biomarker to one subgroup; thus, there may be a range of values for a biomarker for which a clear assessment cannot be provided; in such a case, one or more further lineage biomarker(s) may be used. Preferably, however, as indicated above, a reference enables the assessment to be made for each and every value of a biomarker or set of biomarkers which may be measured. As the skilled person understands, the specific value of a lineage reference may depend on the assessment intended and on parameters thereof.

As indicated herein above, a lineage reference may in particular be derived from at least one pre-defined lineage subgroup, the term “pre-defined group” relating to a group of cells with known status with regard to the assessment. Thus the reference group may e.g. be a group of cells for which lineage status is known. The population of cells in a reference group preferably comprises a plurality of cells, e.g. at least 100, preferably 1,000, more preferably 10,000, even more preferably 100,000, cells. Typically, the cells used for providing the lineage reference and the cancer cells to be allocated are of the same species and, more preferably, of the same lineage. The reference applicable for an individual lineage subgroup may vary depending on various parameters such as lineage, number of subgroups, and other parameters known to the skilled person. Reference amounts can, in principle, be calculated for a population of cells based on the average or mean values for a given parameter such as biomarker amount by applying standard statistical methods.

In a preferred embodiment, the lineage reference is a fixed lineage reference, the term “fixed” lineage relating to a lineage excluding cycling (i.e. dividing) cells. Markers of dividing cells are known in the art, so it is possible to remove cycling cells from a pool of analyzed cells. In a preferred embodiment, cycling cells are identified and excluded as described herein in the Examples; in a further preferred embodiment, a fixed lineage reference is provided as described herein in the Examples.

The term “determining” as used herein refers to semiquantitative or quantitative determination of a biomarker referred to herein. Determining the amount of a biomarker may be carried out by any technique which allows for establishing a measure of quantity of a biomarker in a semiquantitative or quantitative manner. Suitable techniques depend on the molecular nature and the properties of the biomarkers and are discussed elsewhere herein in more detail.

Typically, the amount of a biomarker can be determined by determining a complex of the analyte with a detection compound, in particular an antibody or fragment thereof, i.e. in an immunoassay. Said determining of a complex of the analyte may be performed in any format deemed appropriate by the skilled person, in particular a sandwich, competition, or other assay format. Said assays will develop a signal which is indicative for the amount of a biomarker. Preferably, the lineage biomarker is determined on a single cells level, so determining may e.g. be immunostaining of cells followed by analysis by fluorescence activated cell sorting (FACS). More preferably, the lineage biomarker is determined in a nucleic acid based assay, such as hybridization or, more preferably, sequencing, in particular single-cell sequencing. Thus, the amount of a biomarker may be determined by detecting the amount of molecular species of the biomarker, or of fragments thereof. Preferably, the biomarker is a methylation status of at least three genes in a cell. In accordance, determining a lineage biomarker may also be determining the methylation status of at least three methylation sites, e.g. a CpG site, in a cell. However, also other lineage biomarkers may be envisaged, such as promoter occupancy of a pre-determined promoter, chromatin structure of a biomarker gene, and the like. Also, a biomarker is preferably determined on a single-cell level, preferably in order to allow providing a percentage of cells in a pre-determined subpopulation. A lineage biomarker may, however, also enable providing such percentage without single-cell measurement; e.g. a value of an average methylation status of a pre-determined methylation site in a population of cells may allow direct determination of the fractions of cells in the respective subgroups. Preferably, the lineage biomarker is determined by single cell sequencing and/or methylation analysis, both preferably as described herein in the Examples. Also, in a preferred embodiment, bulk determination (as opposed to single-cell determination) of expression of at least one biomarker may be performed, as described herein in Example 3 and in [49].

The term “comparing” as used herein encompasses comparing the determined amount for a lineage biomarker as referred to herein to a lineage reference. It is to be understood that comparing as used herein refers to any kind of comparison made between the value for the amount with the reference. However, it is to be understood that preferably identical types of values are compared with each other, e.g., if an absolute amount is determined, the reference shall also be an absolute amount, if a relative amount is determined, the reference shall also be a relative amount, etc. The term comparing also encompasses comparing a calculated score with a suitable reference score. The comparison may be carried out manually or computer assisted. The value of the amount and the reference can be, e.g., compared to each other and the said comparison can be automatically carried out by a computer program executing an algorithm for the comparison. As set forth above, it is also envisaged to calculate a score based on the amounts of the biomarkers, in particular a single score, and to compare this score to a reference score. The calculated score preferably combines information on the amounts of a plurality of biomarkers. Moreover, in the score, the biomarkers may be weighted in accordance with their contribution to the establishment of the differentiation, wherein the weighting factor of the individual biomarkers may be different. The score can be regarded as a classifier parameter for the assessing as set forth herein. In particular, it may enable providing the assessment based on a single score. How to elect a suitable reference score is well known in the art. Preferably, the comparing is performed by pseudotime analysis, preferably using a helper algorithm such as “ptalign”, as described herein in the Examples.

The present intention also relates to a method for providing a lineage reference for evaluating cancer cells, said method comprising
(A) providing at least three lineage biomarkers of non-cancer cells of the same lineage as said cancer cells, and
(B) providing said at least three lineage biomarkers as at least one lineage reference for evaluating cancer cells.

The method for providing, preferably, is an in vitro method. Moreover, it may comprise steps in addition to those explicitly mentioned above. For example, further steps may relate, e.g., to determining a number of candidate lineage biomarkers for step (A). Moreover, one or more of said steps may be assisted or performed by automated equipment. The method may also be computer-implemented, i.e. may be an in silico method.

The term “providing” is used herein in a broad sense including any and all means and methods of making the indicated information or item available. Thus, the lineage biomarker may be provided as a lineage biomarker information, preferably tangibly embedded on a data carrier. In accordance with the description herein above, the lineage biomarker may be a lineage biomarker known as such to the skilled person, or the lineage biomarker may be newly identified, preferably as specified herein above. In view of the above, providing a lineage biomarker as a lineage reference may be identifying the lineage biomarker as suitable as a lineage biomarker for evaluating cancer cells and providing a reference thereof as specified herein above, e.g. a reference value, a reference range, or a score. Preferably, providing a lineage biomarker as a lineage reference further comprises verifying that the lineage biomarker is also determinable in cancer cells. Preferably, a multitude of lineage biomarkers and/or lineage references is provided. Also preferably, at least one lineage reference is provided per lineage subpopulation.

The term “evaluating” cancer cells, as referred to herein, relates to providing information which may be deemed medically and/or diagnostically relevant. Preferably, evaluating cancer cells is allocating cancer cells to lineage subpopulations, more preferably is determining lineage subpopulations in a sample of said cancer cells, all as specified herein above. Also preferably, evaluating cancer cells is identifying a subject susceptible to treatment with a wnt modulator. Also preferably, evaluating cancer cells is prognosing cancer. Also preferably, evaluating cancer cells is aiding in establishing a treatment plan for a subject, e.g. including the treatment steps or combinations thereof described herein above.

The present invention further relates to a method for evaluating cancer cells comprising
(a) determining at least three lineage biomarkers in a sample of said cancer cells;
(b) comparing said at least three lineage biomarkers determined in step (a) to a lineage reference; and thereby
(c1) evaluating cancer cells.

The method for providing, preferably, is an in vitro method. Moreover, it may comprise steps in addition to those explicitly mentioned above. For example, further steps may relate, e.g., to cancer cell for determining lineage biomarkers for step (a). Moreover, one or more of said steps may be assisted or performed by automated equipment.

As indicated herein above, evaluating cancer cells may in particular be allocating cancer cells to lineage subpopulations, preferably determining lineage subpopulations in a sample of said cancer cells; identifying a subject susceptible to treatment with a wnt modulator; and/or prognosing cancer, preferably glioma.

The present invention also relates to a database comprising
(I) at least three lineage biomarkers as a cancer cell lineage reference; allocated to
(II) at least one cancer cell subpopulation identifier.

The term “database”, as used herein, refers to a collection of data which may be physically and/or logically grouped together. Accordingly, the database preferably comprises an allocation of at least three lineage biomarkers as a cancer cell lineage reference to at least one cancer cell subpopulation identifier, thus, the database enables allocating a lineage biomarker determined in cancer cells to an assessment result, i.e. allocation to lineage subgroups. As the skilled person understands from the description herein above, a cancer cell lineage reference may also be one or more scores derived from one or more lineage reference(s). The database, in an embodiment, comprises further data, such as upper and/or lower detection limits, references for further lineage biomarkers and/or lineage references, in particular those described herein above, data relevant for plausibility checks, and the like. In a further embodiment, the database comprises data on one or more determining methods to use, lot-specific data, e.g. for calibrator samples, and the like. In an embodiment, the database may be implemented in a single data storage medium or in physically separated data storage media being operatively linked to each other. Preferably, the database comprises a data collection on a suitable storage medium, in an embodiment tangible embedded thereon. Moreover, the database preferably further comprises a database management system. The database management system preferably is a network-based, hierarchical or object-oriented database management system. Furthermore, the database may be a federal or integrated database. Also preferably, the database will be implemented as a distributed (federal) system, e.g. as a Client-Server-System. Preferably, the database is structured as to allow a search algorithm to compare a test data set with the data sets, in particular the references, comprised by the data collection. Specifically, by using such an algorithm, the database can be searched for similar or identical data sets being indicative for a subpopulation or effect as set forth above (e.g. a query search). Thus, preferably, if a data set fulfilling the comparison criteria as detailed elsewhere herein can be identified in the database, the test data set will be associated with the said lineage subgroup. Consequently, the information obtained from the database can be used, e.g., as a reference for the methods described elsewhere herein.

The term “subpopulation identifier” is used herein in a broad sense including any and all data enabling identification of a lineage subpopulation in a data set, such as a database. Thus, the subpopulation identifier may e.g. be a designation of a subpopulation, such as a conventional name, which may e.g. be based on morphological or physiological properties of the subpopulation, such as “activated”, “quiescent” and the like. The subpopulation identifier may also comprise a designation of a biomarker, such “X positive”, with X being a lineage biomarker. The identifier may, however, also be an alphanumeric or numeric identifier; in such case, the database preferably comprises a further allocation of the aforesaid identifier to a description, e.g. of lineage subpopulation properties. The term “cancer cell subpopulation identifier” is understood by the skilled person in view of the description herein above. The cancer cell subpopulation identifier may, in principle, be identical to a subpopulation identifier as specified herein above; thus, e.g. a subpopulation identifier from a non-cancer lineage subpopulation may be used. The cancer cell subpopulation identifier may, however, also be cancer cell subpopulation specific, e.g. may comprise descriptive terms such as “favorable prognosis”, “highly metastatic”, “wnt inhibitor insensitive”, and the like. Preferably, the database further comprises, allocated to the aforesaid data, data relating to the evaluation of cancer cells allocated to the subpopulation, in particular an identification of susceptibility to a particular treatment, a treatment recommendation, and/or a prognosis.

The present invention also relates to a method for treating a glioma in a subject, said method comprising
(I) administering a wnt modulator to said subject, and
(II) thereby treating and/or preventing said glioma in said subject.

The method for treating is an in vivo method comprising administration of a wnt modulator to a subject, as specified herein above. The method may, however, comprise further steps such as evaluating or having evaluated cancer cells from said subject.

Thus, the method may in particular be method for treating a glioma in a subject, said method comprising
(0) evaluating or having evaluated cancer cells from said subject;
(I) based on the result of the evaluation in step (0), administering a wnt modulator to said subject, and
(II) thereby treating said glioma in said subject.

The present invention also relates to a use of a lineage biomarker of non-cancer cells for cancer stratification, wherein said use preferably is an ex vivo use.

The present invention also relates to a data carrier comprising, preferably tangibly embedded, the database of the present invention.

The present invention also relates to a device comprising the data carrier of the present invention and/or, preferably tangibly embedded, the database of the present invention.

As used herein, the term “device” includes any and all contraptions comprising the components specified. Preferably, the device is adapted to perform a method as specified herein. Thus, the device preferably comprises (i) an analysis unit comprising a means for determining a lineage biomarker, and, operatively connected thereto (ii) an evaluation unit comprising tangibly embedded executable instructions for performing a method as specified herein and, preferably, a database as specified herein above. Typical means for determining a lineage biomarker are known to the skilled person and have been discussed herein elsewhere. An evaluation means may be any means capable of providing the analysis as specified; preferably, the evaluation means is a data processing means, such as a microprocessor, a handheld device such as a mobile phone, or a computer. How to link the means in an operating manner will depend on the type of means included into the device. Preferably, the means are comprised by a single device. Said device may accordingly include (i) an analyzing unit for the measurement a lineage biomarker and a (ii) computer unit for processing the resulting data for the evaluation. Preferably, the instructions and interpretations are comprised in an executable program code comprised in the device, such that, as a result of determination an evaluation of cancer cells may be provided. The results may be given as output of raw data which need interpretation by a technician. Preferably, the output of the device is, however, processed, i.e. evaluated, raw data, the interpretation of which does not require a technician. Further typical devices comprise analyzing units/devices (e.g., biosensors, arrays, solid supports coupled to ligands specifically recognizing binding, Plasmon surface resonance devices, NMR spectrometers, mass-spectrometers etc.) or evaluation units/devices referred known to the skilled person. Preferably, the device further comprises a memory unit, preferably comprising a database as specified herein above.

The invention further discloses and proposes a computer program including computer-executable instructions for performing the method according to the present invention in one or more of the embodiments enclosed herein when the program is executed on a computer or computer network or a device as specified herein. Specifically, the computer program may be stored on a computer-readable data carrier. Thus, specifically, one, more than one or even all of method steps as indicated above may be performed by using a computer or a computer network, in an embodiment by using a computer program. Thus, the present invention in particular proposes a computer program comprising instructions which, when the program is executed by a computer, cause the computer to carry out a method as specified herein above; and to a computer-readable medium comprising instructions which, when executed by a computer, cause the computer to carry out a method as specified herein above; to a computer-readable data carrier having stored thereon the computer program as specified herein above; and to a data carrier signal carrying the computer program as specified herein above. The present invention also relates to a data processing apparatus, device, or system comprising means for carrying out performing the method according to the present invention; to a data processing apparatus, device, or system comprising a processor configured to perform the method according to the present invention.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier.

Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the invention proposes and discloses a modulated data signal which contains instructions readable by a computer system or computer network, for performing the method according to one or more of the embodiments disclosed herein.

Preferably, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

In view of the above, the following embodiments are particularly envisaged:

Embodiment 1: A modulator of wnt activity (wnt modulator) for use in treating a glioma in a subject.

Embodiment 2: The wnt modulator for use of embodiment 1, wherein cancer cells of said glioma were allocated to a multitude of lineage subpopulations.

Embodiment 3: The wnt modulator for use of embodiment 1 or 2, wherein cancer cells of said glioma were evaluated by the method according to any one of embodiments 28 to 39.

Embodiment 4: The wnt modulator for use of embodiment 2 or 3, wherein said multitude of lineage subpopulations comprises a quiescent subpopulation, an activation subpopulation, and a differentiation subpopulation.

Embodiment 5: The wnt modulator for use of any one of embodiments 1 to 4, wherein said wnt modulator is an inhibitor of wnt activity (wnt inhibitor).

Embodiment 6: The wnt modulator for use of embodiment 5, wherein said wnt inhibitor is a compound providing a notum polypeptide (Genbank Acc No. NP_848588.3).

Embodiment 7: The wnt modulator for use of embodiment 4 or 5, wherein at least 25%, preferably at least 35%, more preferably at least 50% of tumor cells of said glioma are from an activation subpopulation or a differentiation subpopulation and wherein said wnt inhibitor is a compound providing a notum polypeptide, a porcupine polypeptide (Genbank Acc No. NP_073736.2), secreted frizzled-related protein 1 (sFRP1, Genbank Acc No. NP_003003.3), or is an anti-dickkopf-related protein 1 (DKK1, Genbank Acc No. NP_036374.1) antibody.

Embodiment 8: The wnt modulator for use of any one of embodiments 5 to 7, wherein said subject is not planned to undergo chemotherapy.

Embodiment 9: The wnt modulator for use of any one of embodiments 5 to 8, wherein said treatment is conservative or palliative treatment.

Embodiment 10: The wnt modulator for use of any one of embodiments 5 to 9, wherein said subject is a subject of at least 50 years, preferably at least 60 years, more preferably at least 70 years, most preferably at least 80 years, of age.

Embodiment 11: The wnt modulator for use of any one of embodiments 5 to 10, wherein said treating comprises administration of said wnt inhibitor after diagnosis of said glioma.

Embodiment 12: The wnt modulator for use of any one of embodiments 5 to 11, wherein said treating comprises administration of said wnt inhibitor until surgical resection of said glioma or a part thereof, preferably until at most 12h, preferably at most 1d, more preferably at most 2d, before surgical resection of said glioma of a part thereof.

Embodiment 13: The wnt modulator for use of any one of embodiments 1 to 4, wherein said wnt modulator is an activator of wnt activity (wnt activator).

Embodiment 14: The wnt modulator for use of embodiment 13, wherein said wnt activator is an inhibitor of sFRP1, preferably is WAY-316606 (5-(phenylsulfonyl)-N-4-piperidinyl-2-(trifluoromethyl)-benzenesulfonamide, CAS No. 915759-45-4).

Embodiment 15: The wnt modulator for use of embodiment 13, wherein at least 25%, preferably at least 35%, more preferably at least 50% of cancer cells of said glioma are from a quiescent subpopulation and wherein said wnt activator is an inhibitor of sFRP1.

Embodiment 16: The wnt modulator for use of any one of embodiments 13 to 15, wherein said treating further comprises administration of chemotherapy and/or radiotherapy.

Embodiment 17: The wnt modulator for use of any one of embodiments 13 to 16, wherein said treating comprises administration of said wnt activator during and/or after surgical resection, preferably in combination with chemotherapy and/or radiotherapy.

Embodiment 18: The wnt modulator for use of any one of embodiments 1 to 17, wherein said glioma is an astrocytoma, preferably a glioblastoma, more preferably a glioblastoma multiforme.

Embodiment 19: Use of a wnt modulator for the manufacture of a medicament for treating glioma.

Embodiment 20: A method for determining whether a subject suffering from a glioma is susceptible to treatment with a wnt modulator, said method comprising
(a) determining at least three lineage biomarkers in a sample of cancer cells of said subject,
(b) comparing said at least three lineage biomarkers determined in step (a) to a lineage reference; and
(c) identifying a subject susceptible to treatment with a wnt modulator based on the result of comparing step (b).

Embodiment 21: The method of embodiment 20, wherein said method further comprises step (b1) allocating said cancer cells of step (a) to a multitude of lineage subpopulations.

Embodiment 22: The method of embodiment 20 or 21, wherein said multitude of lineage subpopulations comprises a quiescent subpopulation, an activation subpopulation, and a differentiation subpopulation.

Embodiment 23: The method of any one of embodiments 20 to 22, wherein said subject is identified to be susceptible to treatment with a wnt inhibitor in case at least 25%, preferably at least 35%, more preferably at least 50% of cancer cells of said glioma are allocated to the activation subpopulation or the differentiation subpopulation.

Embodiment 24: The method of any one of embodiments 20 to 23, wherein said subject is identified to be susceptible to treatment with a wnt activator in case at least 25%, preferably at least 35%, more preferably at least 50% of cancer cells of said glioma are allocated to the quiescent subpopulation.

Embodiment 25: A method for providing a lineage reference for evaluating cancer cells, said method comprising
(A) providing at least three lineage biomarkers of non-cancer cells of the same lineage as said cancer cells, and
(B) providing said at least three lineage biomarkers as at least one lineage reference for evaluating cancer cells.

Embodiment 26: The method of embodiment 25, wherein said method is a method of providing a multitude of lineage biomarkers and/or lineage references.

Embodiment 27: The method of embodiment 25 or 26, wherein lineage biomarkers for a multitude of non-cancer cell lineage subpopulations are provided and wherein at least one lineage reference is provided for each of said non-cancer cell lineage subpopulations.

Embodiment 28: A method for evaluating cancer cells comprising
(a) determining at least three lineage biomarkers in a sample of said cancer cells;
(b) comparing said at least three lineage biomarkers determined in step (a) to a lineage reference; and thereby
(c1) evaluating cancer cells.

Embodiment 29: The method of embodiment 28, wherein said evaluating cancer cells is allocating cancer cells to lineage subpopulations, preferably is determining lineage subpopulations in a sample of said cancer cells.

Embodiment 30: The method of embodiment 28 or 29, wherein said evaluating cancer cells is identifying a subject susceptible to treatment with a wnt modulator.

Embodiment 31: The method of any one of embodiments 28 to 30, wherein said lineage biomarker is a lineage biomarker determined in a method according to any one of embodiments 25 to 27.

Embodiment 32: The method of any one of embodiments 28 to 31, wherein said lineage reference is a reference derived from a lineage analysis of non-cancer cells of the same cell lineage as the cancer cells.

Embodiment 33: The method of any one of embodiments 28 to 32, wherein evaluating cancer cells is prognosing cancer.

Embodiment 34: The method of any one of embodiments 28 to 33, wherein said cancer cells are cancer cells from a glioma and said reference is a reference derived from a lineage analysis of non-cancer cells of the glial lineage.

Embodiment 35: The method of any one of embodiments 28 to 34, wherein said cancer cells are cancer cells from an astrocytoma, preferably a glioblastoma, more preferably a glioblastoma multiforme.

Embodiment 36: The method of embodiment 35, wherein said lineage reference is a reference derived from a lineage analysis of non-cancer cells of the astrocyte lineage.

Embodiment 37: The method of embodiment 35 or 36, wherein said lineage biomarker is expression of at least one gene selected from Table 1 or, in a preferred embodiment, from Table 2.

Embodiment 38: The method of any one of embodiments 35 to 37, wherein said lineage biomarker is methylation status of at least one gene.

Embodiment 39: The method of any one of embodiments 20 to 38, comprising determining a multitude of lineage biomarkers, preferably at least 5, more preferably at least 10, still more preferably at least 20, lineage biomarkers.

Embodiment 40: A database comprising
(I) at least three lineage biomarkers as a cancer cell lineage reference; allocated to
(II) at least one cancer cell subpopulation identifier.

Embodiment 41: The database of embodiment 40, further comprising at least one treatment recommendation allocated to said at least one cancer cell subpopulation identifier.

Embodiment 42: The database of embodiment 40 or 41, wherein said lineage biomarker was obtained by the method according to any one of embodiments 25 to 27.

Embodiment 43: The database of any one of embodiments 40 to 42, wherein said database is tangibly embedded on a data carrier.

Embodiment 44: A data carrier comprising the database according to any one of embodiments 40 to 43.

Embodiment 45: A device comprising the data carrier of embodiment 44 and/or, preferably tangibly embedded, the database according to any one of embodiments 40 to 43.

Embodiment 46: The device of embodiment 45, further comprising a processor and, preferably tangibly embedded, instructions which, when performed on the processor, cause the device to perform at least step (b) of the method according to any one of embodiments 20 to 24 and 28 to 39.

Embodiment 47: A method for treating a glioma in a subject, said method comprising
(I) administering a wnt modulator to said subject, and
(II) thereby treating said glioma in said subject.

Embodiment 48: Use of a lineage biomarker of non-cancer cells for cancer stratification.

Embodiment 49: The use of embodiment 48, wherein said use is a use in a method according to any one of embodiments 20 to 24 and 28 to 39.

Embodiment 50: The use of embodiment 48 or 49, wherein said use is an ex vivo use, preferably an in silico use.

Embodiment 51: The wnt modulator for use of any one of embodiments 2 to 18, wherein said cancer cells of said glioma were allocated to said multitude of lineage subpopulations by determining at least one lineage biomarker being expression of at least one gene selected from Table 1 or, in a preferred embodiment, from Table 2.

All references cited in this specification are herewith incorporated by reference with respect to their entire disclosure content and the disclosure content specifically mentioned in this specification.

### FIGURE LEGENDS

FIG. 1. Construction of the adult neural stem cell (NSC) lineage and the extraction of steady cell states across pseudotime by single cell RNA sequencing (scRNA-seq). A. scRNA-seq of 14793 ventricular-subventricular zone (vSVZ) NSCs and their progeny from 6 wt-mice ([30]; [31]; [29]) captures vSVZ astrocytes/NSCs (qNCS1, qNSC2, aNSC1 and aNSC2) as well as transit amplifying progenitors (TAPs) and neuroblasts (NBs). B. Diffusion pseudotime records lineage transitions along the differentiation continuum found in NSCs upon exclusion of actively cycling cells (light gray background). C. According to the diffusion pseudotime, the maintenance and dynamics of the SVZ lineage can be reduced to the transitions between three stable cell states: quiescence(Q), activation(A) and differentiation(D). D-E. We derive a 654-gene pseudotime-predictive geneset to facilitate their identification across datasets. F-G-H. Gene ontology analysis on the extracted genesets robustly represents QAD cell state-related cellular processes.

FIG. 2. Pseudotime alignment by ptalign resolves neoplastic QAD cell states in human glioblastoma multiforme (GBM) subjected to scRNA-seq. A. Schematic representation of the ptalign workflow. B-C. Correlations were computed between query cells and the pseudotime-binned reference for the supplied geneset (B), with each query cell's pseudotime being derived from the correlation dynamics with respect to the reference. A neural network was trained to predict a given cell's pseudotime from the reference-reference correlation matrix (C), and fed the dynamics of individual query cells to determine their aligned pseudotime (D). D. ptalign-predicted QAD cell states and pseudotime for human GBM in an integrated UMAP of 4 patient derived xenograft (PDX) tumors.

FIG. 3. Pseudotime alignment by ptalign enables clinically relevant patient stratification based on lineage occupancy. A. UMAP of 51 primary patient GBM scRNA-seq datasets, here greyscaled by publication source, clusters tumors by patient origin. B. ptalign QAD cell state proportions of 51 GBMs represented in a cell state ternary. Tumors' ptalign correlation heat maps demonstrate diverse correlation structure related to predicted lineage cell states. C. ptalign QA-D cell state barcharts representing intra-tumor pseudolineage heterogeneity. Total- and cycling-cell numbers are indicated in circles below; 1: total cells, 2: cycling cells. D. TCGA tumors embedding in GSVA PCA captures lineage heterogeneity and identifies known and novel tumor classes. E. The novel GBM classes QAD and QA demonstrate the best and worst survival outcomes in the TCGA cohort, respectively.

FIG. 4. Tumor methylome is predictive of neoplastic QAD cell states. A-B. Euclidean distances across highly variable methylation sites and genes demonstrates significant positive correlation in an individual tumor (A) and across the cohort (B) in Wu et. al. 2020 [17]. C. PCA embedding of 83 GBMs with matched gene expression (microarray or RNA-seq based) and methylation array separates tumors by their pseudolineage classes. D. ElasticNet regression predicts GSVA cell state scores from methylation data, achieving holdout pearson correlations of 0.22, 0.53, 0.57 and 0.57 for Q, A and D, as well as CC-scores, respectively. E. Regression and 90% CI on a (n=28) holdout set for ElasticNet predictions on methylation data of GBM-QAD signatures.

FIG. 5. Wnt-antagonist over-expression stalls GBM lineage progression and leads to increased quiescence and overall survival. A. sFRP1- and NOTUM-overexpressing mice (n=6; n=5) exhibit significantly increased survival compared to Wnt-reporter controls (n=6). B. ptalign cell states in scRNA-seq of SFRP1- and NOTUM-overexpressing PDX samples reveals significantly increased quiescence including in the astroQ population, which is an early-pseudotime subset of Q, with a concomitant reduction in activation. C. ptalign pseudotime densities for SFRP1- and NOTUM-overexpressing and control Wnt-reporter PDX samples highlight cells' shift out of activation towards quiescence without disturbing differentiation. D. Immunohistochemistry highlights extensive morphological changes (arrow heads exemplifying neuron like vs astrocyte like morphology) in SFRP1-overexpressing compared to control Wnt-reporter PDX, in the mouse cortex. E. as in A., but Kaplan-Meier curves only of mice reaching end point post injection for n=6 mice in control and SFRP1 cohorts. Significance was assessed by log-rank test. the dashed line denotes termination of experiment.

FIG. 6. Stratification with low numbers of biomarkers. A. Per-tumor pseudobulk mean proportion of DRAXIN A^{nc}-state expression (nc: non-cycling). Statistical significance was determined by two-sided t-test with Benjamini-Hochberg correction. B. Stratification using only biomarkers ID3 (quiescence), DLL3 (activation), and MYT1L (differentiation); with expr.: taking into account only cells which express the markers, n: number of cells in analysis.

FIG. 7. SFRP1 Inhibitor Treatment. A. Exemplary microscopy photograph of cells treated with DMSO (control, upper row) or SFRP1 inhibitor WAY-316606 (lower row), as described in Example 8; mCherry: fluorescence of the cytoplasmic mCherry RFP (all tumor cells within human brain organoid), KI-67: immunohistochemistry stain of KI-67 (proliferation nuclear marker, i.e. activation); B. quantification of KI-67+(proliferating) tumor cells/mm³, three sections/human brain organoids, in 2 human brain organoids. and averaging of A. over several frameData is expressed as a density metric reflecting the detected KI-67+/mCherry+ tumor cells in the images normalized to the organoid volume.

The following Examples shall merely illustrate the invention. They shall not be construed, whatsoever, to limit the scope of the invention.

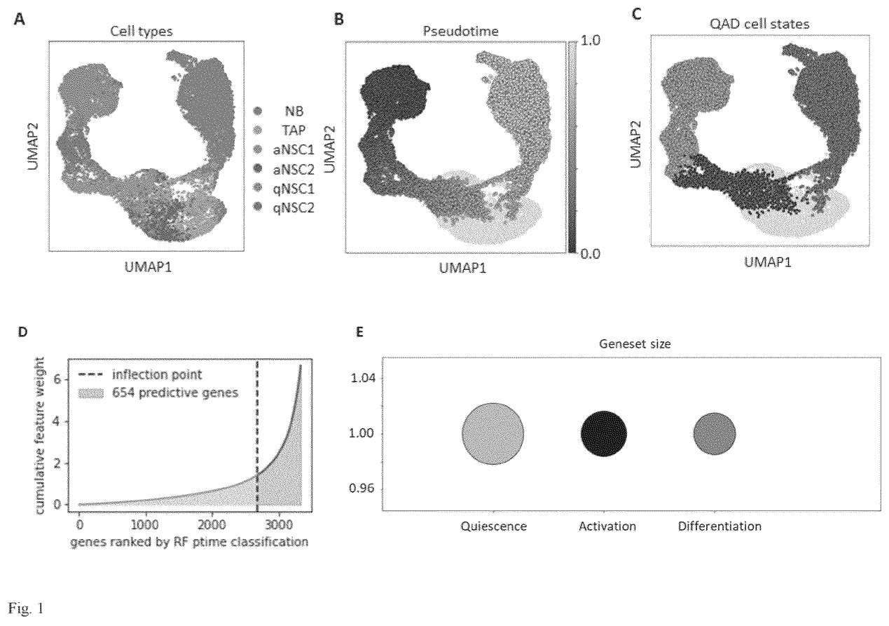

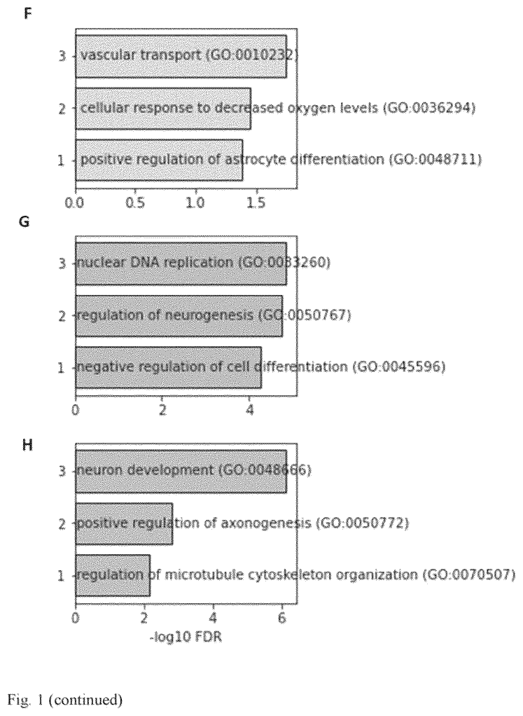

### Example 1: Shared State Transitions and Expression Dynamics in Mouse- and Human NSCs (FIG. 1)

The celltypes and lineage dynamics of the mouse SVZ are well studied [1-4]. NSCs are born in the early postnatal brain and remain in a dormant, quiescent state until niche signals instruct their activation, proliferation, and subsequent differentiation. Sampling at a single timepoint, scRNAseq of SVZ NSCs and their progeny robustly captures Astrocytes, NSCs, transit amplifying progenitors (TAPs), as well as neuroblasts (NBs) and neurons, along with the transitions between them. Here, we construct a 14,793-cell reference lineage from 6 WT SVZ replicates and record the lineage transitions by a diffusion pseudotime (FIG. 1A, B). We exclude actively cycling cells from all datasets for which we derive a lineage pseudotime as the cycle itself represents a branching event in pseudotime and disrupts the otherwise consistently 1D differentiation continuum found in NSCs. Comparing to 10,022 cells from 4 human cortical organoids, the transfer of celltype labels by integration with an organoid reference [5] and their mapping to the above NSC lineage celltypes, highlights an NSC lineage composed of shared lineage states while its pseudotime reproduces the ordering between them. Indeed, the expression dynamics of ID3, a marker of quiescence; ASCL1, a marker of activation; and DCX, a marker of differentiation, between human and mouse lineage pseudotimes reinforces the conserved principles of stem-cell activation in these two models.

Exclusion of cycling cells in the SVZ reference: Cellcycle scoring was carried out following the scanpy ‘180209_cell_cycle’ example notebook (notebook.community/theislab/scanpy_usage/180209_cell_cycle/cell_cycle) with the there-supplied genes. Filtering for 1:1 orthologs we scored 51 G2M- and 42 S-phase genes. We set a G2M-score cutoff at 0.1 to determine cycling activity, which was consistent with expression of known cellcycle markers including MKI67, TOP2A, and UBE2C (not shown). We identified and excluded 2.769 cycling cells, leaving 12.024 noncycling cells for which we computed a noncyling lineage pseudotime as described above.

The maintenance and dynamics of the SVZ lineage can be reduced to the transitions between three stable states: Quiescence (Q), Activation (A), and Differentiation (D). We are able to extract these states from the SVZ lineage pseudotime (FIG. 1), essentially splitting the NSC population into quiescent and active populations and lumping the former with the dormant niche astrocytes (astroQ). Importantly, these states are able to be identified from pseudotime alone, and we derive a 242-gene pseudotime-predictive geneset to facilitate their identification across datasets (cf. Table 1).

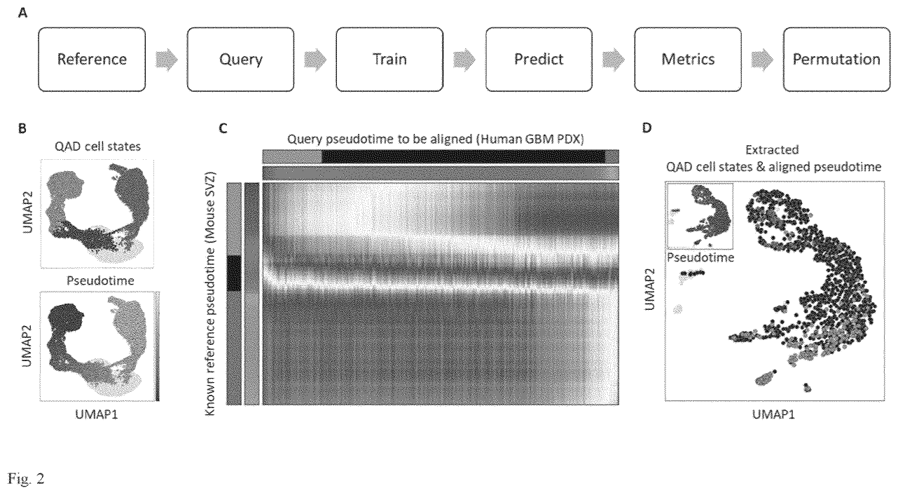

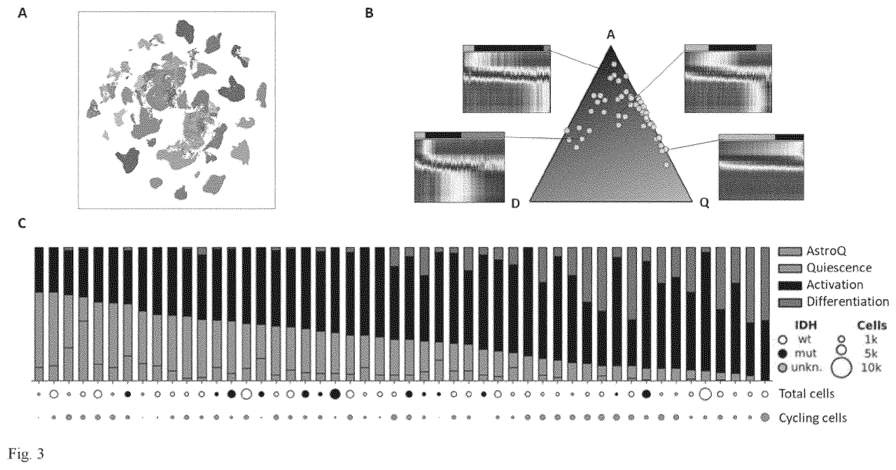

### Example 2: Ptalign Projects GBM Cells Along a NSC Pseudolineage (FIG. 2)

Glioma stem-cells (GSCs) have long been known to resemble their healthy counterparts in the adult brain, and represent the prime suspect for the tumor cell-of-origin [6-10]. The advent of scRNA-seq brought with it the ability to identify celltype-specific expression programs, enabling the identification of GSC meta-modules [11] and delivering a statistical framework for the interpretation of GSC expression patterns. Yet such approaches fail to consider the relation of tumor cells to each other and provide limited means for the functional interpretation of tumor processes. To address these limitations, we have developed ptalign (FIG. 2) to project individual tumor cells onto a reference lineage by pseudotime alignment. In this way, tumor cell states are linked to those from the reference, and the comparison of individual cells by their pseudotime can refer to contextual knowledge available for a given reference.

ptalign is a lightweight software with built-in multithreading capabilities and reporting of permutation statistics to assess the significance of a particular pseudotime alignment. The tool requires a query and reference counts matrix, as well as the reference cell's pseudotime and a geneset comprising pseudotime-predictive genes. Briefly, correlations are computed between query cells and the pseudotime-binned reference for the supplied geneset (FIG. 2), with each query cell's pseudotime being derived from the correlation dynamics with respect to the reference. To this end, a neural network is trained to predict a given cell's pseudotime from the reference-reference correlation matrix, and fed the dynamics of individual query cells to determine their ‘aligned’ pseudotime. Lineage states, in this case Q, A, and D, can then be assigned based on each cell's aligned pseudotime, and we refer to their relative ratios and composition as the query's pseudolineage.

Alignment quality metrics are derived by the dynamic time warping (DTW) of reference- and query-cells in equivalent pseudotime bins, including the determination of an optimal alignment path maximizing DTW correlations, rewarding higher correlation spread (ie. cellstate specificity) in the DTW matrix, and scoring the narrowness of the main diagonal. These metrics are used in a permutation framework, whereby expression-matched genesets are derived from the reference counts matrix and used to conduct pseudotime alignment. A permutation p-value is reported for the various alignment metrics, communicating the ability for the supplied geneset to explain the pseudotime dynamics for a given reference-query pairing.

We used ptalign to determine the lineage pseudotime for a patient derived (patient pseudonym: T6) xenograft (PDX) sequenced 5 months post injection (mpi) by Smart-seq3. Q-A-D cellstate scoring by AUCell indicated the presence of all three lineage states in the xenograft (data not shown), and these were consistently identified by ptalign (FIG. 2D). The T6 pseudolineage was dominated by tumor cells in the Activation state, which were complemented by a large Quiescence and smaller Differentiation population, respectively. We separately generated T6 tumor allografts (PDA) by injecting tumor spheres into human cortical organoids and found that PDA pseudolineages recapitulated those of the PDX, hinting at cell-intrinsic fating of GSCs (not shown).

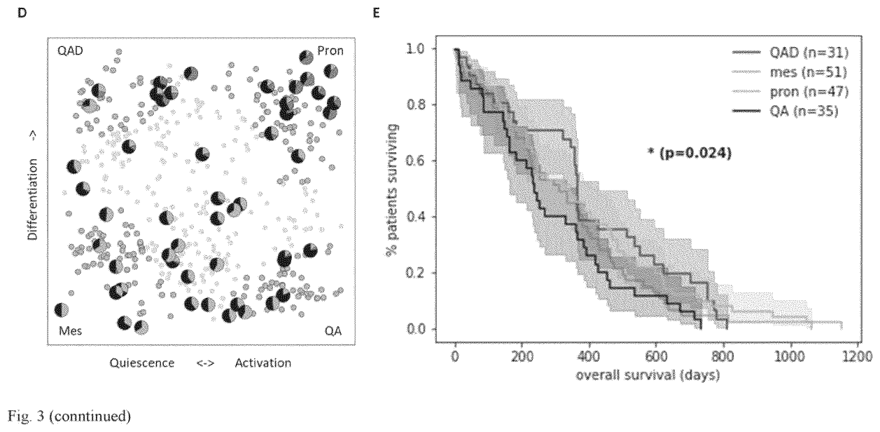

### Example 3: Stratification by GBM Pseudolineage Informs Clinically Relevant Tumor Classes (FIG. 3)

Curious to assess the pseudolineage heterogeneity among GBMs, we compiled scRNA-seq data from 57 published primary GBMs [5, 11-15]. Malignant cells were identified by inferCNV [16] and tumors required to contain at least 500 cells. Unsurprisingly, a UMAP embedding of these tumors clustered them by their patient origin (FIG. 3A). Comparison of these tumors in the more standardized ptalign framework revealed 1) a high degree of intra-tumoral pseudolineage heterogeneity, dominated by Quiescence and Activation states (FIG. 3B) non-significant pseudotime alignments in 12 tumors, with these enriched in IDH-mutants and lower-grade gliomas. Interestingly, GBM pseudolineages followed the same progression as SVZ cells' QAD AUCell scores (FIG. 3B, top) with Q-D transitioning populations equally absent in both ternary, while, similarly, every tumor exhibited Quiescence and Activation cells, with Differentiation apparently dispensable (FIG. 3B)—these observations together hinting at lineage constraint inherent in the development of GBM.

The 45 remaining, significantly Q-A-D, tumors showed a high degree of pseudolineage heterogeneity (FIG. 3C). Available metadata were sparse but gave no indication of a significant association between tumor pseudolineage and IDH mutation status, tumor location, patient gender, or patient age. Proportions of cycling cells varied between tumors and correlated with the degree of Activation. ptalign determined a single tumor to have a large population of dormant astrocyte-like (astroQ, FIG. 3C) cells, which supported its origins in astrocytoma as reported in the metadata.

We sought to determine the association between GBM pseudolineages and patient outcomes. As no survival data were available from our tumor cohort we used the scRNA-seq Q, A, and D populations to derive a 257-gene GBM QAD geneset (cf. Table 2) which we used as a proxy to stratify by GBM pseudolineage in the TCGA IDH-wt GBM cohort (n=367, including microarray and RNA-seq data) and those from Wu et al. [17](n=45, RNA-seq). Nonparametric geneset scoring by GSVA decomposed TCGA GBMs into a four-quadrant embedding characterized by different combinations of Q, A, and D scores (FIG. 3D). We confirmed the efficacy of pseudolineage estimation from bulk data by GSVA through the pseudolineage-driven embedding of our scRNA-seq GBMs. Comparison to the existing TCGA tumor classification [18] highlighted two pre-existing tumor classes (Mesenchymal, Proneural) as well as two novel (termed QAD, QA) groups (FIG. 3D). All comparisons between tumor groups spanning the Q-to-A transition indicated significantly lower survival for the A-high group, highlighting the role of the cycling population in tumor progression. Intriguingly, the novel tumor groups QAD and QA exhibited the best and worst survival outcomes, respectively (FIG. 3E); while a higher degree of Quiescence was protective in a Cox-hazard model, consistent with the QAD and mesenchymal classes' improved survival outcomes despite their otherwise divergent pseudolineages (not shown). Thus, Q-A-D state scoring in bulk tumor samples was sufficient to demonstrate different disease outcomes relating to GBM pseudolineages.

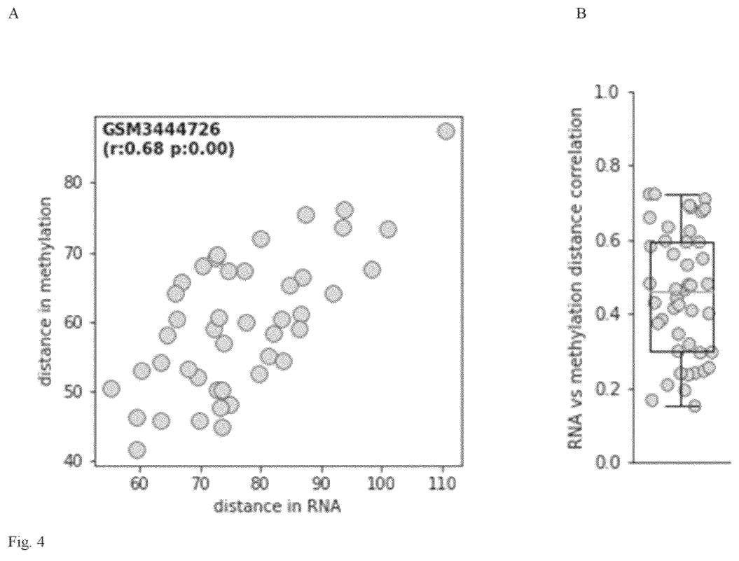

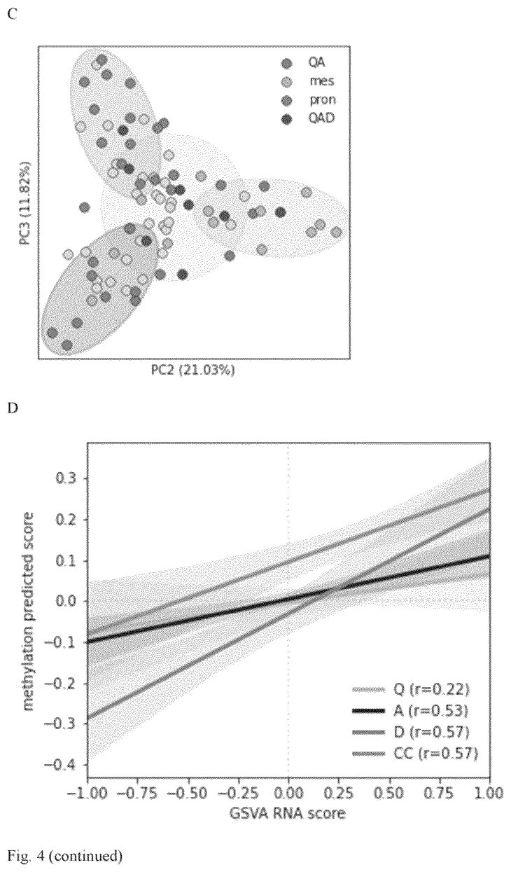

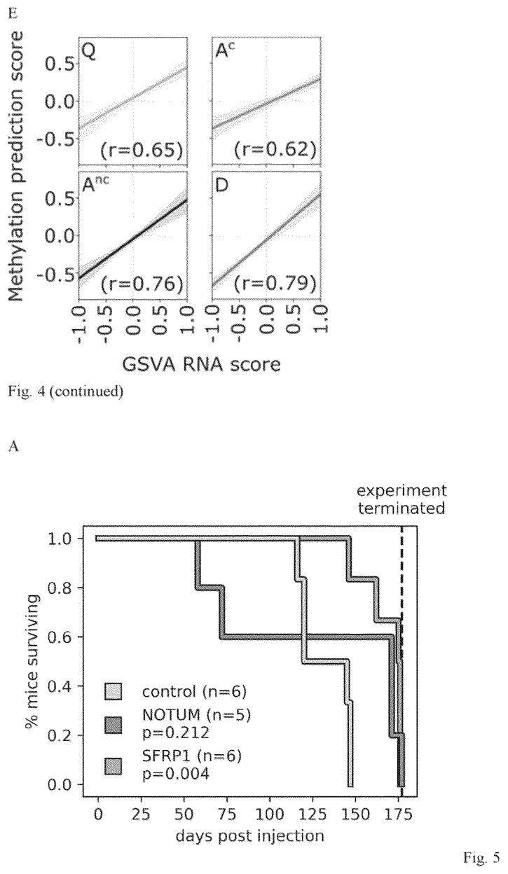

### Example 4: Tumor Methylome is Predictive of Q-A-D States (FIG. 4)

As GBM Q-A-D states were associated with significant differences in patient outcome, we sought to identify a strategy to infer a tumor's pseudolineage in a time- and cost-effective manner. This way, we could leverage GBM pseudolineages to monitor disease progression and tumor evolution, while potentially informing personalized treatments. Glioma patient material is routinely assessed by methylation array (eg. Illumina EPIC 850k) for patient stratification in a clinical context, and we decided to assay the ability for a patient's methylation status to predict the Q-A-D classes identified in FIG. 2.

Returning to our TCGA and Wu et al. [17] IDH-wt GBM cohort, we identified 90 tumors with matched RNA and methylation data. PCA embedding of these tumors by highly-variable probes separated tumors by their pseudolineage classes (FIG. 4C) along PC2 and PC3. The first principle component, on the other hand, resolved two groups with vastly different survival: all but one of the PC1-hi patients remained alive at the time of measurement. It is likely that these patients do not represent bona fide high-grade glioma but rather belong to auxiliary methylation groups identified eg. in [19].

RNA GSVA scores were predicted from tumor's methylomes after confirming a baseline of correlation between RNA- and methylation-distances in the Wu et al. cohort [17](FIG. 4A). Tumors were split into train- and test-groups, scaled to zero-mean and unit variance, and a cross-validated gridsearch ElasticNet regression used to predict GSVA Q, A, and D scores individually. Chained regression, whereby previous predictions are incorporated into future ones (ie. where the Q-prediction could inform the D-prediction) did not improve regression accuracy. The trained predictors achieved good performance measured by the pearson coefficient of the holdout data, with better predictions at the extremes of the respective genesets and a consistent underestimation of GSVA-scores. Thus, these data demonstrate the feasibility of predicting tumor pseudolineage classes using routine methylation array data for diagnostics. Future work remains to combine the Q, A, and D predictors from FIG. 3D into an ensemble classifier for tumor pseudolineage class, as well as an exploration into the flip-side of this analysis: assessing the ability to predict methylation features (eg. PCA coordinates) from the RNA features.

More so than with RNA, we were able to use the recently published single-cell methylomes of SVZ populations in [20] to demonstrate that tumor Quiescence is not linked to the terminal differentiation into astrocytes as suggested by [11, 21]. Identifying SVZ populations' differentially-methylated regions (DMRs) and lifting their coordinates to the human genome, we compared 2308 probes from regions with significantly lower methylation in either NSCs, astros, or oligos. Tumor methylation profiles across DMRs and between celltypes were consistently closest to that of NSCs, with Q-high GBMs exhibiting more NSC- and astro-like methylomes than their Q-low counterparts. A similar trend was observed for D-high and -low populations and their methylation at oligo-specific DMRs. Taken together, these data suggest that GBM Quiescence is closer to that of quiescent NSCs over niche astrocytes, suggesting their role in maintaining the GSC pool. Future work along this avenue might consider neuron-specific CH methylation profiles measured in SVZ methylomes and present on the methylation arrays to identify an association between the Differentiation states of GBMs and the SVZ.

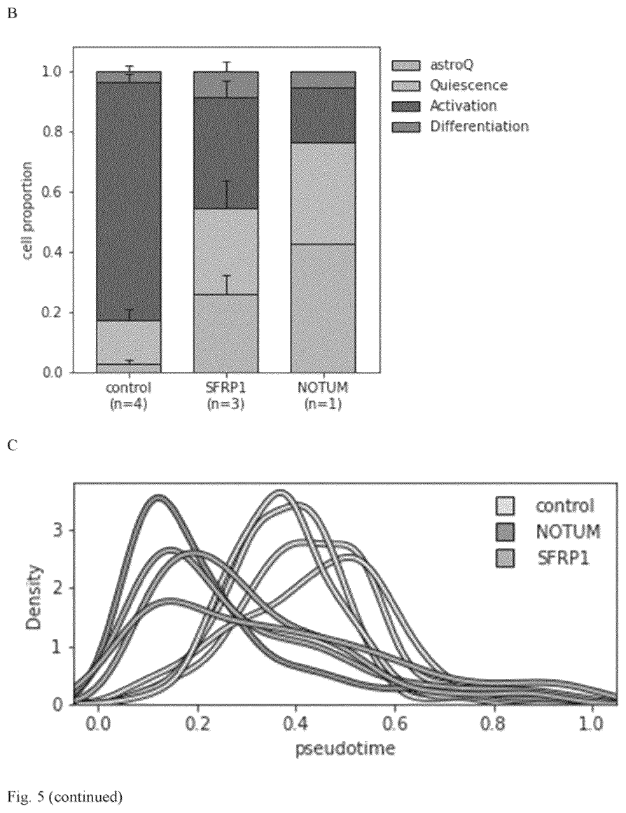

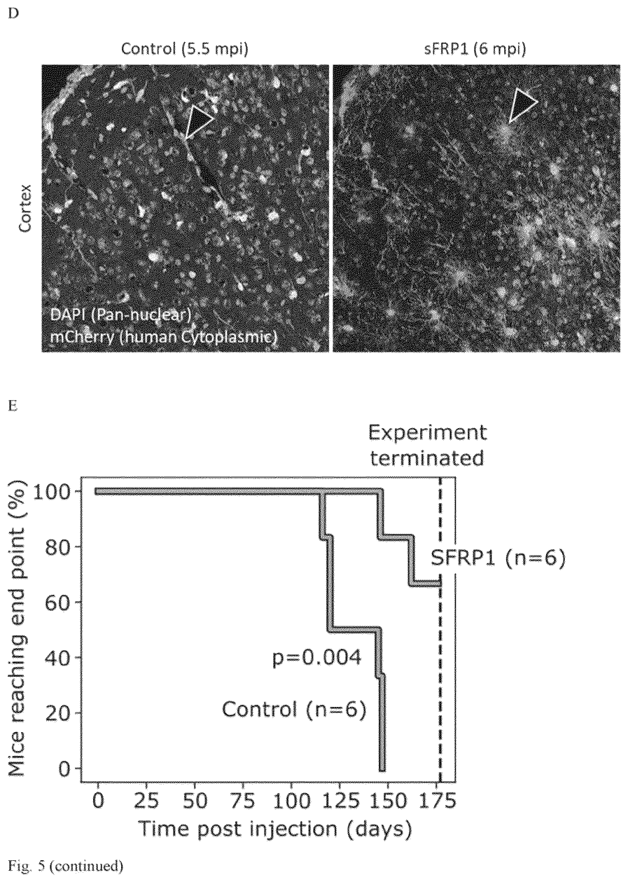

### Example 5: Directed Modulation of GBM Pseudolineages by Intervention in Wnt Signaling (FIG. 5)

Finally, having demonstrated the clinical relevance of GBM pseudolineages and the feasibility of their identification in a time- and cost-effective manner using methylation arrays, we aimed to interfere with the malignant lineage progression by intervening in the Wnt signaling pathway. Wnt signaling plays a critical role in the maintenance of the SVZ niche and regulates the activation state of adult NSCs [4]. Employing a lentiviral TCF/Lef-EGFP reporter in our T6 PDX and PDA models as well as a TCF/Lef:H2B-EGFP line, we observed strict regulation of Wnt activity at SVZ state transitions in vivo which was observed to be lost in the tumor both by scRNA-seq and by IHC. Thus we postulated that intervention in the Wnt signaling pathway might direct tumor cell fates consistent with their role in the healthy NSC lineage.

We identified the secreted Wnt antagonist SFRP1 as being implicated in the Q-A transition in the healthy SVZ. Single-cell methylomes from [20] implicate a switch in SFRP1 methylation between astrocytes and NSCs; and expression data from our lineage reference demonstrates a sharp peak in expression at the Q-A transition. Similarly, [22] reports a role of SFRP1 in regulating the activation of human NSCs, and data from our own lab [4] implicates its most-conserved paralog, SFRP5, in the activation of murine NSCs. Following these evidences and in order to target Wnt-active tumor cells, we cloned human SFRP1 downstream of the TCF/Lef promoter and generated T6 PDA and PDX tumors as outlined previously.

SFRP1-overexpression (OE) led to a stark and reproducible increase in tumor Quiescence, both at a transcriptional and morphological level. Wnt modulation via SFRP1 induction in PDX GBMs led to a significant increase in overall survival (FIG. 5A, E), with treated mice surviving 1 month longer on average before the experiment was terminated. Subsequent scRNA-seq of control and SFRP1 PDXs highlighted a significant increase in tumor Quiescence, particularly toward the dormant astroQ state, with a concomitant reduction in the Activation state and a relative increase in cycling cells (FIG. 5B). These effects were consistent yet weaker in our 2-week PDA replicates (not shown). Movement out of Activation and toward Quiescence is demonstrated by the tumors' aligned pseudotimes (FIG. 5C), but appears to be a one-way transformation as tumor Differentiation was not affected. Subsequent integration of PDX replicates and analysis of differential UMAP occupancy highlights the overrepresentation of astrocytic- and underrepresentation of neuronal-transcripts predicated on the treatment status, which was supported by DEseq. In line with the molecular readout of the effect of SFRP1-OE, tumor IHC revealed a dominant astrocyte-like morphology characterized by a highly connected appearance with extensive tumor processes (FIG. 5D). Taken together, overexpression of the soluble Wnt antagonist SFRP1 induced a remarkable shift in tumor cell transcription and morphology by acting on the Q-A transition, consistent with its expression profile in the healthy SVZ. Future directions in this vein include the development of deeper insight into the mechanism of action of SFRP1-OE, its effect on the homeostatic mouse brain, and an assessment of the morphological properties characterizing the phenotype.

### Example 6: Methods

### 6.1 Cells, Patient Tumors and Tumor Sphere Culture

Primary tumor samples were received from University Hospital Ulm upon obtaining informed consent prior to surgery. Experiments involving patient tumor biopsies was carried out in accordance with the Decleration of Helsinki and were approved by the ethics committees of University Hospital Ulm, Clinic for Neurosurgery Gunzburg and Heidelberg University, Medical Faculty Mannheim. All tumor specimens were examined by a neuropathologist to ensure that the tumors met GBM criteria defined by WHO. Upon arrival, fresh tissues were immediately dissociated using Brain Tumor Dissociation Kit (P) and expanded in culture.

Tumor spheres were maintained in serum-free Neurobasal A medium supplemented with B27, heparin (2 μg/ml) and the stem mitogens EGFP (20 ng/ml) and bFGF (20 ng/ml) at 5% CO₂ and 37° C. For passaging, tumor spheres were enzymatically dissociated into single cells using Accutase when sphere size reached approximately 100 μm in diameter once in 1-2 weeks.

### 6.2 Lentiviral Transduction

To allow sorting of human GBM cells, cells were infected with the 7TGC (Addgene plasmid #24304) lentiviral vector at a multiplicity of infection (MOI) of 5. Expression of the transduction marker (mCherry) was confirmed by FACS analysis. Second-generation, self-inactivating, replication deficient lentiviral particles were produced, purified and titrated as described previously ([32]).

### 6.3 Injection of GBM Cells

For injection of lentivirally transduced primary GBM cells into human brain organoids, 3×10⁴ cells were cultured as single cell suspension overnight. Newly formed spheres were resuspended in 1 μl of Differentiation Medium with vitamin A, loaded into a NanoFil syringe and injected into the core of 2 month-old organoids under a dissection microscope. Tumor bearing organoids were maintained in Differentiation Medium with vitamin A on an orbital shaker (70 RPM) for 15 days at 5% CO₂ and 37° C.

### 6.4 Animal Experiments

### Mouse Strains

Male Fox Chase SCID Beige mice (CB17. Cg-Prkdc^{scid}Lyst^{bg-J}/Crl) were purchased from Charles River and were used to generate human-mouse xenograft tumors. Experimental mice had ad libitum access to food and water and were housed in specific pathogen-free, light (12 hr day/night cycle), temperature (21° C.) and humidity (50-60% relative humidity) controlled conditions. All procedures conform to the institutional guidelines of the DKFZ and were approved by the ethical authorities, Regierungsprasidium Karlsruhe, Germany.

### Orthotopic Injection of GBM Cells

For ortotopic injection of lentivirally transduced primary GBM cells into the mouse brain, 5×10⁵ cells were cultured as single cell suspension overnight. Newly formed spheres were resuspended in 2 μl of Matrigel, loaded into a NanoFil syringe and stereotactically injected into the striatum (2.5 mm lateral to the bregma at a depth of 3.0 mm) of 8-10 week-old Fox Chase SCID-Beige mice under anesthesia. Tumor growth was longitudinally monitored by magnetic resonance imaging at the Small Animal Imaging Center at the DKFZ. Upon reaching termination criteria, mice were sacrificed and brains were collected after transcardial perfusion. For perfusion, mice were anesthetized by intraperitoneal injection of 800 μl of perfusion solution. After opening the thoracic cavity exposing heart, transcardial perfusion was carried out with 10 ml of ice cold HBSS.

### 6.5 FACS Analysis and Sorting

For isolation of the xenografted human GBM cells, tumor bearing mouse brains were dissected and single cell suspension was prepared using Brain Tumor Dissociation Kit (P) and gentleMACS Octo Dissociator with Heaters (Miltenyi) according to manufacturer's instructions. mCherry+ cell population was index sorted into 384 well-plates (Eppendorf Lobind). Microplates containing cell lysates were briefly centrifuged, snap frozen on dry ice and were stored at −80° C. Sytox Blue (Life Technologies, 1:1000) was used in all experiments as a dead cell indicator. Index sorting of single cells as well as sorting of bulk samples was carried out using a 100 micron nozzle at a BD FACSAria II or BD FACSAria Fusion at the Flow Cytometry Core Facility at the DKFZ.

6.6 Single Cell Library Preparation with Smart-Seq3

Single cell RNA-sequencing libraries from patient-derived xenografts were prepared using Smart-seq3 platform as previously described (Hagemann-Jensen, Nature Biotechnology, 2020) with minor modifications. The protocol was automated and miniaturized by incorporating liquid handling platforms including Mosquito HV (STPLabtech), Mantix (Formulatrix) and Viaflo 384 (Integra). Briefly, plates were incubated at 72° C. for 10 min to facilitate lysis and denaturation of secondary structures in the RNA. Lysed cells were subjected to reverse transcription in 2 μl using Maxima H-minus reverse transcriptase (Thermo Scientific), an oligo(dT) primer and a template-switching oligonucleotide encompassing an 8-bp unique molecular identifier (IDT). In a randomly selected set of wells, we included ERCC Spike-ins (Ambion) at a 1:2500000 dilution. Full length cDNAs were amplified for 22 cycles of PCR using KAPA HiFi DNA polymerase (KAPA Biosystems). cDNA samples were purified with Ampure XP beads at a 1:0.8 ratio and cDNA quality in randomly selected 10 wells/plate was assessed on a High Sensitivity Bioanalyzer chip (Agilent). cDNA concentrations were quantified using Quant-it PicoGreen dsDNA Assay kit (Thermo Scientific) and Synergy LX multi-mode microplate reader (Biotek). cDNAs were normalized to 250-500 pg μl⁻¹. 100-200 pg of cDNA per sample was used for tagmentation in 1.2 μl using Illumina XT DNA sample preparation kit. Libraries were finally amplified for 11 cycles of PCR in 4 μl using custom-designed Nextera index primers containing 8-bp index barcode sequences with a minimal Levenshtein distance of 4 as previously published (Buenrostro et al., Nature, 2015). Samples were purified with Ampure XP beads at a 1:0.8 ratio and DNA quality in randomly selected 10 wells/plate was assessed on a High Sensitivity Bioanalyzer chip (Agilent). Libraries were quantified as mentioned above and normalized libraries were equimolarly pooled and purified one last time at a 1:0.8 ratio. Prior to sequencing, final library concentration was determined using Qubit dsDNA High Sensitivity Assay kit (Thermo Scientific) and Qubit fluorometer (Invitrogen) and the average fragment size was calculated on a High Sensitivity Bioanalyzer chip (Agilent). Libraries with 1% spiked-in PhiX control (Illumina) were sequenced at the 75-bp paired end on a high output flow cell using an Illumina NextSeq550 instrument at a sequencing depth of ~1 M reads per cell at the sequencing open lab at the DKFZ.

### 6.7 Illumina Infinium MethylationEPIC Array

Genomic DNA isolation from primary patient-derived GBM cells was carried out using QIAamp DNA Micro kit (Qiagen). The Illumina Infinium MethylationEPIC kit was used to analyze the DNA methylation status at >850000 5′CpG islands per sample according to the manufacturer's instructions. The assay was run at the Genomics and the Proteomics Core Facility of the German Cancer Research Center (DKFZ) Heidelberg.

### 6.8 Ptalign for Pseudotime Alignment

In sequence alignment, the position of one query sequence in a larger reference sequence is ascertained by scoring local sequence similarity until an optimal match is found. We employ a conceptually similar strategy to infer a given query cell's optimal positioning in the space of a reference pseudotime trajectory in a method we call ptalign. The ptalign program requires four central inputs: a query counts table, a reference counts table, a reference pseudotime mapping, and a set of genes capturing the reference pseudotime trajectory. Query cells are then placed along the reference pseudotime according to the dynamic of the correlation of their gene expression along the trajectory genes. Briefly, reference cells are binned into equal-sized bins along pseudotime and the mean expression taken per bin. Pearson correlations are computed between the query cell transcriptomes and the pseudotime-binned reference over the supplied trajectory genes. Resulting correlation matrices are optionally normalized by row (reference bin), then scaled by column (query cell) to a value between 0 and 1. These scaled correlations represent a given cell's distance to different parts of the reference pseudotime, and in the next step is reduced to a single value representing pseudotime. To this end, ptalign uses the supplied reference counts and reference pseudotime to compute a reference-reference correlation matrix and train a small multi-layer perceptron (MLP) network to predict the known reference pseudotime from the correlation dynamic in the supplied matrix. A 5-fold cross-validated grid search is performed over a number of relevant parameters to find the best network hyperparameters for the supplied reference and geneset. Then, query cell's pseudotimes are fed into the trained network to predict pseudotime values. Cellstates are optionally inferred based on cutoffs derived from the reference pseudotime. The quality of an assigned pseudotime is determined via the computation of a dynamic time warping (DTW) matrix over the pseudotime-binned reference and the equally binned query. Counts are log-normalized and pearson correlations computed for all combinations of reference and query bins. A matrix traceback is computed by dynamic programming to determine a path of maximal correlation through the DTW matrix. ptalign performance is estimated by comparing the length of the DTW traceback, the average correlation along the traceback, and by extracting the narrowness-parameter from a parabolic fit to the mean correlation along the diagonals of the DTW matrix. These three metrics are compared to reference-query matrices computed according to equally sized and equivalently expressed permuted genesets, to determine an empirical p-value relating the ability for the supplied trajectory geneset to explain the query's dynamics relative to random genesets.

### Example 7: Stratification with Low Numbers of Biomarkers

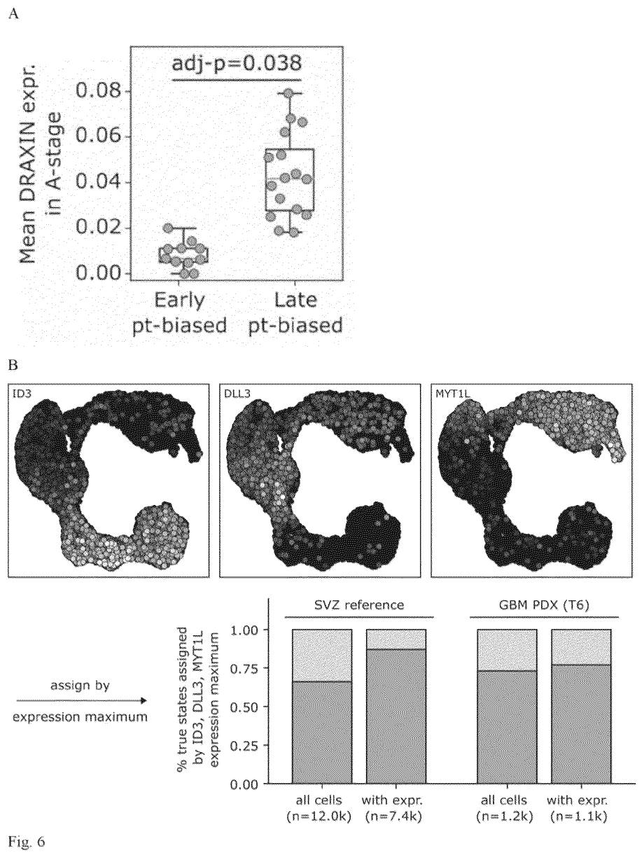

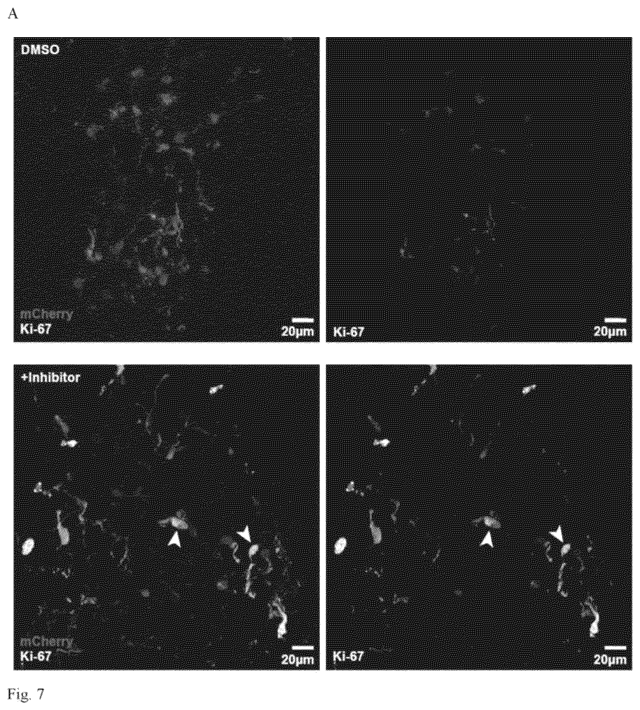

We compared expression in A^{nc} cells in 11 early-pseudotime (pt) biased and 16 late-pt biased tumors determined by the relative prevalence of dormant astrocyte-like Q-cells or D-cells, respectively. This analysis identified the secreted Wnt antagonist DRAXIN as a reliable predictor of tumor pseudolineage, being consistently higher-expressed in late-pt biased tumors compared to early-pt biased ones (FIG. 6A).

Also, stratification using only three biomarkers was performed “by hand”, i.e. without using ptalign, by assigning each cell to a QAD stage by its maximum expression of the three biomarkers ID3, DLL3, and MYT1L. This approach achieves 66% accuracy among all SVZ cells and 87% when considering only those which express these markers (FIG. 6B). In a GBM PDX (line T6) >70% accuracy was achieved in both cases (FIG. 6B).

### Example 8: SFRP1 Inhibitor Treatment

Lentivirally transduced GBM cells for SFRP1 overexpression were injected into day 116-old human brain organoids and cultured in brain Organoid Differentiation Medium with Vitamin A. Injected organoids were maintained for 15 days on an orbital shaker (70 RPM) at 5% CO2 and 37° C. After 15 days, organoids were transferred to culturing medium supplemented with 25 μM of SFRP1 inhibitor WAY-316606 or 25 μM DMSO as a control. Tumour-bearing organoids were maintained for 2 more days and then fixed with 4% formaldehyde for 1 hour at 4° C. on a roller. Fixed organoids were first placed in 10% sucrose in PBS until tissues sank, then transferred to 30% sucrose in PBS. Organoids were then embedded in OCT compound (Tissue-Tek). 10 μm-thick organoid sections were cut with a Leica CM1950 cryostat and placed on poly-l-lysine coated slides.

### 8.1 Immunohistochemistry and IMAGING

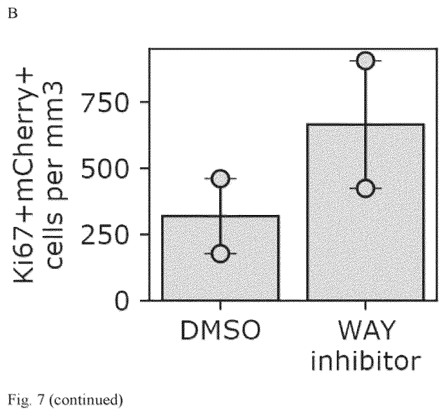

Sections were washed with PBS and blocked with 3% Horse Serum and 0.3% Triton X-100 in PBS (blocking buffer) for 1 hour at room temperature. Sections were then incubated with the primary antibodies rabbit anti-RFP (1:1000, Rockland Immunochemicals), mouse anti-Ki-67 (1:400, Merck Millipore) in blocking buffer overnight at 4° C. Sections were then washed with 0.3% Triton X-100 in PBS and incubated in blocking buffer for 15 minutes at room temperature. Alexa fluor antibodies (1:400) were diluted in blocking buffer and incubated for 1 hour at room temperature. Sections were then washed in PBS and mounted in Fluoromount-G mounting medium with DAPI (ThermoFisher Scientific). Slides were stored at 4° C. before imaging. Images and tilescans were acquired using a Leica SP8 or Zeiss 780D confocal microscope. Representative frames are shown in FIG. 7A.

### 8.2 Image Analysis and Quantification

Confocal images of human brain organoids injected with GBM m-Cherry- and sFRP1-expressing cells that were treated with DMSO or sFRP1 inhibitor. Organoids were stained against Ki67 and mCherry and Ki67+/mCherry+ cells were quantified. To this end, a 2D Cellpose model was trained from scratch and applied to maximum projections of the Ki67 channel to generate instance segmentation masks. Object features were then extracted using scikit-image. Inferred objects were filtered by size and circularity to exclude mis-segmented entities and debris. The tumor origin of segmented cells was determined by assessing background corrected mean fluorescence intensities of the 2D integrated mCherry signal. The count of detected Ki67+/mCherry+ cells was then normalized to the imaged organoid volume yielding a density metric representing cycling tumor cells in both WAY inhibitor treated and DMSO control samples. Results are shown in FIG. 7B. In conclusion, inhibition of SFRP1, e.g. by WAY-316606, drastically increases the number of proliferating cells in the GBM tumor model, making a higher number of cells accessible to chemotherapy.

### REFERENCES

[1] Lim, D. A. and Alvarez-Buylla, A., 2016. The adult ventricular-subventricular zone (V-SVZ) and olfactory bulb (OB) neurogenesis. Cold Spring Harbor perspectives in biology, 8(5), p.a018820.
[2] Obernier, K. and Alvarez-Buylla, A., 2019. Neural stem cells: origin, heterogeneity and regulation in the adult mammalian brain. Development, 146(4), p.dev156059.
[3] Cebrian-Silla, A., Nascimento, M. A., Redmond, S. A., Mansky, B., Wu, D., Obernier, K., Rodriguez, R. R., Gonzalez-Granero, S., Garcia-Verdugo, J.M., Lim, D. A. and Alvarez-Buylla, A., 2021. Single-cell analysis of the ventricular-subventricular zone reveals signatures of dorsal and ventral adult neurogenesis. Elife, 10, p.e67436.
[4] Kalamakis, G., Brune, D., Ravichandran, S., Bolz, J., Fan, W., Ziebell, F., Stiehl, T., Catali-Martinez, F., Kupke, J., Zhao, S. and Llorens-Bobadilla, E., 2019. Quiescence modulates stem cell maintenance and regenerative capacity in the aging brain. Cell, 176(6), pp. 1407-1419.
[5] Bhaduri, A., Andrews, M. G., Mancia Leon, W., Jung, D., Shin, D., Allen, D., Jung, D., Schmunk, G., Haeussler, M., Salma, J. and Pollen, A. A., 2020. Cell stress in cortical organoids impairs molecular subtype specification. Nature, 578(7793), pp. 142-148.
[6] Galli, R., Binda, E., Orfanelli, U., Cipelletti, B., Gritti, A., De Vitis, S., Fiocco, R., Foroni, C., Dimeco, F. and Vescovi, A., 2004. Isolation and characterization of tumorigenic, stem-like neural precursors from human glioblastoma. Cancer research, 64(19), pp. 7011-7021.
[7] Singh, S. K., Clarke, I.D., Terasaki, M., Bonn, V. E., Hawkins, C., Squire, J. and Dirks, P. B., 2003. Identification of a cancer stem cell in human brain tumors. Cancer research, 63(18), pp. 5821-5828.
[8] Lee, J. H., Lee, J. E., Kahng, J. Y., Kim, S. H., Park, J. S., Yoon, S. J., Um, J. Y., Kim, W. K., Lee, J. K., Park, J. and Kim, E. H., 2018. Human glioblastoma arises from subventricular zone cells with low-level driver mutations. Nature, 560(7717), pp. 243-247.
[9] Alcantara Llaguno, S., Sun, D., Pedraza, A.M., Vera, E., Wang, Z., Burns, D. K. and Parada, L. F., 2019. Cell-of-origin susceptibility to glioblastoma formation declines with neural lineage restriction. Nature neuroscience, 22(4), pp. 545-555.
[10] Bhaduri, A., Di Lullo, E., Jung, D., Muller, S., Crouch, E. E., Espinosa, C. S., Ozawa, T., Alvarado, B., Spatazza, J., Cadwell, C. R. and Wilkins, G., 2020. Outer radial glia-like cancer stem cells contribute to heterogeneity of glioblastoma. Cell stem cell, 26(1), pp. 48-63.
[11] Neftel, C., Laffy, J., Filbin, M. G., Hara, T., Shore, M. E., Rahme, G. J., Richman, A. R., Silverbush, D., Shaw, M. L., Hebert, C. M. and Dewitt, J., 2019. An integrative model of cellular states, plasticity, and genetics for glioblastoma. Cell, 178(4), pp. 835-849.
[12] Wang, Q., Hu, B., Hu, X., Kim, H., Squatrito, M., Scarpace, L., DeCarvalho, A. C., Lyu, S., Li, P., Li, Y. and Barthel, F., 2017. Tumor evolution of glioma-intrinsic gene expression subtypes associates with immunological changes in the microenvironment. Cancer cell, 32(1), pp. 42-56.
[13] Yuan, J., Levitin, H.M., Frattini, V., Bush, E. C., Boyett, D.M., Samanamud, J., Ceccarelli, M., Dovas, A., Zanazzi, G., Canoll, P. and Bruce, J. N., 2018. Single-cell transcriptome analysis of lineage diversity in high-grade glioma. Genome medicine, 10(1), pp. 1-15.
[14] Couturier, C. P., Ayyadhury, S., Le, P. U., Nadaf, J., Monlong, J., Riva, G., Allache, R., Baig, S., Yan, X., Bourgey, M. and Lee, C., 2020. Single-cell RNA-seq reveals that glioblastoma recapitulates a normal neurodevelopmental hierarchy. Nature communications, 11(1), pp. 1-19.
[15] Richards, L.M., Whitley, O. K., MacLeod, G., Cavalli, F.M., Coutinho, F. J., Jaramillo, J. E., Svergun, N., Riverin, M., Croucher, D. C., Kushida, M. and Yu, K., 2021. Gradient of developmental and injury response transcriptional states defines functional vulnerabilities underpinning glioblastoma heterogeneity. Nature Cancer, 2(2), pp. 157-173.
[16] inferCNV of the Trinity CTAT Project. github.com/broadinstitute/inferCNV
[17] Wu, Y., Fletcher, M., Gu, Z., Wang, Q., Costa, B., Bertoni, A., Man, K.-H., Schlotter, M., Felsberg, J., Mangei, J., Barbus, M., Gaupel, A.-C., Wang, W., Weiss, T., Eils, R., Weller, M., Liu, H., Reifenberger, G., Korshunov, A., . . . Radlwimmer, B. (2020). Glioblastoma epigenome profiling identifies SOX10 as a master regulator of molecular tumour subtype. Nature Communications, 11(1), 6434. doi.org/10.1038/s41467-020-20225-w
[18] Verhaak, R. G., Hoadley, K. A., Purdom, E., Wang, V., Qi, Y., Wilkerson, M. D., Miller, C. R., Ding, L., Golub, T., Mesirov, J. P. and Alexe, G., 2010. Integrated genomic analysis identifies clinically relevant subtypes of glioblastoma characterized by abnormalities in PDGFRA, IDH1, EGFR, and NF1. Cancer cell, 17(1), pp. 98-110.
[19] Noushmehr, H., Weisenberger, D. J., Diefes, K., Phillips, H. S., Pujara, K., Berman, B. P., Pan, F., Pelloski, C. E., Sulman, E. P., Bhat, K. P. and Verhaak, R. G., 2010. Identification of a CpG island methylator phenotype that defines a distinct subgroup of glioma. Cancer cell, 17(5), pp. 510-522.
[20] Kremer, L. P., Cerrizuela, S., Al Shukairi, M. E., Ellinger, T., Straub, J., Dehler, S., Korkmaz, A., Weichenhan, D., Plass, C., Anders, S. and Martin-Villalba, A., 2022. Single-cell triple-omics uncovers DNA methylation as key feature of sternness in the healthy and ischemic adult brain. bioRxiv.
[21] Tirosh, I., Venteicher, A. S., Hebert, C., Escalante, L. E., Patel, A. P., Yizhak, K., Fisher, J.M., Rodman, C., Mount, C., Filbin, M. G. and Neftel, C., 2016. Single-cell RNA-seq supports a developmental hierarchy in human oligodendroglioma. Nature, 539(7628), pp. 309-313.
[22] Donega, V., van der Geest, A. T., Sluijs, J. A., van Dijk, R. E., Wang, C. C., Basak, O., Pasterkamp, R. J. and Hol, E. M., 2022. Single-cell profiling of human subventricular zone progenitors identifies SFRP1 as a target to re-activate progenitors. Nature communications, 13(1), pp. 1-12.
[23] Jung and Park (2020), Exp Mol Med 5(2):183)
[24] Kierulf-Vieira et al (2016), Exp Cell Res 340: 53
[25] CN 114948933 A
[26] WO 2004/032838 A2
[27] Klevenz et al., Cell Mol Life Sci. 2002, 59: 1993-1998
[28] Zhao et al. (2021) Future Medicinal Chem 13(11), doi.org/10.4155/fmc-2021-0036
[29] Carvajal Ibanez, D., Skabkin, M., Hooli, J., Cerrizuela, S., Gopferich, M., Jolly, A., Volk, K., Zumwinkel, M., Bertolini, M., Figlia, G., Hofer, T., Kramer, G., Anders, S., Teleman, A. A., Marciniak-Czochra, A., & Martin-Villalba, A. (2023). Interferon regulates neural stem cell function at all ages by orchestrating <scp>mTOR</scp> and cell cycle. EMBO Molecular Medicine. doi.org/10.15252/EMMM.202216434
[30] Kalamakis, G., Brune, D., Ravichandran, S., Bolz, J., Fan, W., Ziebell, F., Stiehl, T., Catala-Martinez, F., Kupke, J., Zhao, S., Llorens-Bobadilla, E., Bauer, K., Limpert, S., Berger, B., Christen, U., Schmezer, P., Mallm, J. P., Berninger, B., Anders, S., . . . Martin-Villalba, A. (2019). Quiescence Modulates Stem Cell Maintenance and Regenerative Capacity in the Aging Brain. Cell, 176(6). doi.org/10.1016/j.cell.2019.01.040
[31] Kremer, L. P. M., Cerrizuela, S., Dehler, S., Stiehl, T., Weinmann, J., Abendroth, H., Kleber, S., Laure, A., El Andari, J., Anders, S., Marciniak-Czochra, A., Grimm, D., & Martin-Villalba, A. (2021). High throughput screening of novel AAV capsids identifies variants for transduction of adult NSCs within the subventricular zone. Molecular Therapy. Methods & Clinical Development, 23, 33-50. doi.org/10.1016/j.omtm.2021.07.001
[32] Tiscornia, G., Singer, O., & Verma, I. M. (2006). Production and purification of lentiviral vectors. Nature Protocols, 1(1), 241-245. https://doi.org/10.1038/nprot.2006.37
[33] Alcantara Llaguno, S., Sun, D., Pedraza, A. M., Vera, E., Wang, Z., Burns, D. K., & Parada, L. F. (2019). Cell-of-origin susceptibility to glioblastoma formation declines with neural lineage restriction. Nature Neuroscience, 22(4), 545-555. doi.org/10.1038/s41593-018-0333-8
[34] Bhaduri, A., Di Lullo, E., Jung, D., Müller, S., Crouch, E. E., Espinosa, C. S., Ozawa, T., Alvarado, B., Spatazza, J., Cadwell, C. R., Wilkins, G., Velmeshev, D., Liu, S. J., Malatesta, M., Andrews, M. G., Mostajo-Radji, M. A., Huang, E. J., Nowakowski, T. J., Lim, D. A., . . . Kriegstein, A. R. (2020). Outer Radial Glia-like Cancer Stem Cells Contribute to Heterogeneity of Glioblastoma. Cell Stem Cell, 26(1), 48-63.e6. doi.org/10.1016/j.stem.2019.11.015
[35] Couturier, C. P., Ayyadhury, S., Le, P. U., Nadaf, J., Monlong, J., Riva, G., Allache, R., Baig, S., Yan, X., Bourgey, M., Lee, C., Wang, Y. C. D., Wee Yong, V., Guiot, M.-C., Najafabadi, H., Misic, B., Antel, J., Bourque, G., Ragoussis, J., & Petrecca, K. (2020). Single-cell RNA-seq reveals that glioblastoma recapitulates a normal neurodevelopmental hierarchy. Nature Communications, 11(1), 3406. doi.org/10.1038/s41467-020-17186-5
[35] Fujimura, N., Vacik, T., Machon, O., Vlcek, C., Scalabrin, S., Speth, M., Diep, D., Krauss, S., & Kozmik, Z. (2007). Wnt-mediated Down-regulation of Spl Target Genes by a Transcriptional Repressor Sp5. Journal of Biological Chemistry, 282(2). doi.org/10.1074/jbc.M605851200
[36] Galli, R., Binda, E., Orfanelli, U., Cipelletti, B., Gritti, A., De Vitis, S., Fiocco, R., Foroni, C., Dimeco, F., & Vescovi, A. (2004). Isolation and characterization of tumorigenic, stem-like neural precursors from human glioblastoma. Cancer Research, 64(19), 7011-7021. doi.org/10.1158/0008-5472. CAN-04-1364
[37] Kalamakis, G., Brune, D., Ravichandran, S., Bolz, J., Fan, W., Ziebell, F., Stiehl, T., Catala-Martinez, F., Kupke, J., Zhao, S., Llorens-Bobadilla, E., Bauer, K., Limpert, S., Berger, B., Christen, U., Schmezer, P., Mallm, J. P., Berninger, B., Anders, S., . . . Martin-Villalba, A. (2019). Quiescence Modulates Stem Cell Maintenance and Regenerative Capacity in the Aging Brain. Cell, 176(6). doi.org/10.1016/j.cell.2019.01.040
[38] Lee, J. H., Lee, J. E., Kahng, J. Y., Kim, S. H., Park, J. S., Yoon, S. J., Um, J.-Y., Kim, W. K., Lee, J.-K., Park, J., Kim, E. H., Lee, J.-H., Lee, J.-H., Chung, W.-S., Ju, Y. S., Park, S.-H., Chang, J. H., Kang, S.-G., & Lee, J. H. (2018). Human glioblastoma arises from subventricular zone cells with low-level driver mutations. Nature, 560(7717). https://doi.org/10.1038/s41586-018-0389-3
[39] Neftel, C., Laffy, J., Filbin, M. G., Hara, T., Shore, M. E., Rahme, G. J., Richman, A. R., Silverbush, D., Shaw, M. L., Hebert, C. M., Dewitt, J., Gritsch, S., Perez, E. M., Gonzalez Castro, L. N., Lan, X., Druck, N., Rodman, C., Dionne, D., Kaplan, A., . . . Suva, M. L. (2019). An Integrative Model of Cellular States, Plasticity, and Genetics for Glioblastoma. Cell, 178(4). doi.org/10.1016/j.cell.2019.06.024
[40] Phillips, H. S., Kharbanda, S., Chen, R., Forrest, W. F., Soriano, R. H., Wu, T. D., Misra, A., Nigro, J. M., Colman, H., Soroceanu, L., Williams, P. M., Modrusan, Z., Feuerstein, B. G., & Aldape, K. (2006). Molecular subclasses of high-grade glioma predict prognosis, delineate a pattern of disease progression, and resemble stages in neurogenesis. Cancer Cell, 9(3), 157-173. doi.org/10.1016/j.ccr.2006.02.019
[41] Portela, M., Venkataramani, V., Fahey-Lozano, N., Seco, E., Losada-Perez, M., Winkler, F., & Casas-Tint6, S. (2019). Glioblastoma cells vampirize WNT from neurons and trigger a JNK/MMP signaling loop that enhances glioblastoma progression and neurodegeneration. PLOS Biology, 17(12). https://doi.org/10.1371/journal.pbio.3000545
[42] Rheinbay, E., Suva, M. L., Gillespie, S. M., Wakimoto, H., Patel, A. P., Shahid, M., Oksuz, O., Rabkin, S. D., Martuza, R. L., Rivera, M. N., Louis, D. N., Kasif, S., Chi, A. S., & Bernstein, B. E. (2013). An Aberrant Transcription Factor Network Essential for Wnt Signaling and Stem Cell Maintenance in Glioblastoma. Cell Reports, 3(5). doi.org/10.1016/j.celrep.2013.04.021
[43] Richards, L. M., Whitley, O. K. N., MacLeod, G., Cavalli, F. M. G., Coutinho, F. J., Jaramillo, J. E., Svergun, N., Riverin, M., Croucher, D. C., Kushida, M., Yu, K., Guilhamon, P., Rastegar, N., Ahmadi, M., Bhatti, J. K., Bozek, D. A., Li, N., Lee, L., Che, C., . . . Pugh, T. J. (2021). Gradient of Developmental and Injury Response transcriptional states defines functional vulnerabilities underpinning glioblastoma heterogeneity. Nature Cancer, 2(2), 157-173. doi.org/10.1038/s43018-020-00154-9
[44] Seib, D. R. M., Corsini, N. S., Ellwanger, K., Plaas, C., Mateos, A., Pitzer, C., Niehrs, C., Celikel, T., & Martin-Villalba, A. (2013). Loss of Dickkopf-1 Restores Neurogenesis in Old Age and Counteracts Cognitive Decline. Cell Stem Cell, 12(2). doi.org/10.1016/j.stem.2012.11.010
[45] Siebzehnrubl, F. A., Reynolds, B. A., Vescovi, A., Steindler, D. A., & Deleyrolle, L. P. (2011). The origins of glioma: E Pluribus Unum?Glia, 59(8). doi.org/10.1002/glia.21143
[46] Singh, S. K., Clarke, I. D., Terasaki, M., Bonn, V. E., Hawkins, C., Squire, J., & Dirks, P. B. (2003). Identification of a Cancer Stem Cell in Human Brain Tumors. Cancer Research, 63(18), 5821-5828.
[47] Verhaak, R. G. W., Hoadley, K. A., Purdom, E., Wang, V., Qi, Y., Wilkerson, M. D., Miller, C. R., Ding, L., Golub, T., Mesirov, J. P., Alexe, G., Lawrence, M., O'Kelly, M., Tamayo, P., Weir, B. A., Gabriel, S., Winckler, W., Gupta, S., Jakkula, L., . . . Cancer Genome Atlas Research Network. (2010). Integrated genomic analysis identifies clinically relevant subtypes of glioblastoma characterized by abnormalities in PDGFRA, IDH1, EGFR, and NF1. Cancer Cell, 17(1), 98-110. doi.org/10.1016/j.ccr.2009.12.020
[48] Ziebell, F., Dehler, S., Martin-Villalba, A., & Marciniak-Czochra, A. (2018). Revealing age-related changes of adult hippocampal neurogenesis using mathematical models. Development, 145(1). doi.org/10.1242/dev.153544
[49] Forster et al. (17 Sep. 2023), BioRXiv, doi.org/10.1101/2023.09.15.557713

TABLE 1
Lineage biomarker genes for quiescent, activation, and differentiation
subpopulations in the astrocyte lineage and in glioma

| Quiescence | Genbank Acc No | Activation | Genbank Acc No | Differentiation | GenbankAcc No |
|---|---|---|---|---|---|
| ABHD3 | NP_001295185.1 | ADGRV1 | NP_115495.3 | ABRACL | NP_067066.1 |
| ACAA2 | NP_006102.2 | ANP32B | NP_006392.1 | ADARB2 | NP_061172.1 |
| ACSBG1 | NP_001186306.1 | ARL4C | NP_001269360.1 | ANKS1B | NP_001190999.1 |
| ACSL3 | NP_001341088.1 | ASCL1 | NP_004307.2 | APP | NP_001191230.1 |
| ALDH1L1 | NP_001257294.1 | BTG2 | NP_006754.1 | ARX | NP_620689.1 |
| ALDOC | NP_005156.1 | CCND1 | NP_444284.1 | BASP1 | NP_001258535.1 |
| APPL2 | NP_001238833.1 | CDK6 | NP_001250.1 | BCL11B | NP_001269167.1 |
| AQP4 | NP_001304316.1 | CENPM | NP_001103685.1 | C11orf96 | NP_001138505.1 |
| ASS1 | NP_446464.1 | CORO1C | NP_001263400.1 | CDK5R1 | NP_003876.1 |
| ATP1B2 | NP_001290192.1 | CRLF1 | NP_004741.1 | CELF4 | NP_001340657.1 |
| BAALC | NP_001351803.1 | DCTPP1 | NP_077001.1 | CITED2 | NP_001161861.2 |
| BCAN | NP_940819.1 | DLL1 | NP_005609.3 | CTXN1 | NP_996664.1 |
| CA2 | NP_001280604.1 | DLL3 | NP_982353.1 | DBN1 | NP_001351081.2 |
| CAMK2N1 | NP_061054.2 | DTL | NP_057532.4 | DCBLD1 | NP_775945.1 |
| CHCHD10 | NP_001288268.1 | DTYMK | NP_001307833.1 | DCX | NP_001356299.1 |
| CHST1 | NP_003645.1 | DUT | NP_001317215.1 | DLX2 | NP_004396.1 |
| CHST2 | NP_004258.2 | E2F1 | NP_005216.1 | DLX5 | NP_005212.1 |
| CLDN10 | NP_008915.1 | EGFR | NP_958440.1 | DPYSL3 | NP_001378.1 |
| CLMN | NP_079010.2 | EGR1 | NP_001955.1 | ELAVL3 | NP_115657.2 |
| CLU | NP_001822.3 | FABP7 | NP_001437.1 | ELAVL4 | NP_001311141.1 |
| CMTM5 | NP_001032365.1 | FOS | NP_005243.1 | ENOX1 | NP_001334892.1 |
| CRYM | NP_001363185.1 | FOSB | NP_001107643.1 | FNBP1L | NP_060207.2 |
| CXCL14 | NP_004878.3 | GADD45G | NP_006696.1 | FSCN1 | NP_003079.1 |
| DAAM2 | NP_001188356.1 | GMNN | NP_001238920.1 | FXYD6 | NP_071286.1 |
| DHRS3 | NP_001311299.1 | GNL3 | NP_055181.3 | GAD1 | NP_038473.2 |
| DKK3 | NP_001317149.1 | GSX1 | NP_663632.1 | GAD2 | NP_000809.1 |
| EDNRB | NP_003982.1 | HAT1 | NP_003633.2 | GNG2 | NP_001376639.1 |
| EPHX2 | NP_001243412.1 | HELLS | NP_001276001.1 | IGFBPL1 | NP_001007564.1 |
| EPS8 | NP_004438.3 | HELT | NP_001287711.1 | KIF5C | NP_004513.1 |
| EVA1A | NP_001356453.1 | HES6 | NP_001269363.1 | LMO3 | NP_001230542.1 |
| F3 | NP_001171567.1 | HMGB2 | NP_001124161.1 | LRRC7 | NP_001353771.1 |
| FAM107A | NP_001269642.1 | HMGN5 | NP_110390.1 | LY6H | NP_002338.3 |
| FBXO2 | NP_036300.2 | IER2 | NP_004898.2 | MAP1B | NP_001311184.1 |
| FGFR3 | NP_075254.1 | JUNB | NP_002220.1 | MEX3A | NP_001087194.1 |
| FJX1 | NP_055159.2 | KCNE5 | NP_036414.1 | MLLT11 | NP_006809.1 |
| FXYD1 | NP_001265647.1 | KLF4 | NP_001300981.1 | MPPED2 | NP_001575.1 |
| GABBR2 | NP_005449.5 | LIMA1 | NP_001107019.1 | MTSS1 | NP_001269900.1 |
| GABRB1 | NP_000803.2 | LRRFIP1 | NP_001131024.1 | MYT1L | NP_001316778.1 |
| GABRG1 | NP_775807.2 | LSM2 | NP_067000.1 | NAV3 | NP_055718.4 |
| GJB6 | NP_001103691.1 | LYAR | NP_001139197.1 | NOL4 | NP_001371402.1 |
| GPC5 | NP_004457.1 | MCM2 | NP_004517.2 | NREP | NP_001135948.1 |
| GPR37L1 | NP_004758.3 | MCM3 | NP_001257401.2 | NRXN3 | NP_001258949.1 |
| GRIN2C | NP_001265482.1 | MCM5 | NP_006730.2 | NSG1 | NP_001369157.1 |
| GRM3 | NP_001350451.1 | MCM6 | NP_005906.2 | NSG2 | NP_057064.1 |
| HAPLN1 | NP_001875.1 | MFNG | NP_001159815.1 | PAFAH1B3 | NP_001139411.1 |
| HEPACAM | NP_689935.2 | NBL1 | NP_877421.2 | PBX3 | NP_001317711.1 |
| HOPX | NP_631958.1 | NCALD | NP_001035718.1 | PCDH9 | NP_001305301.1 |
| HSPA2 | NP_068814.2 | NHP2 | NP_001383039.1 | PLCL1 | NP_006217.3 |
| HTRA1 | NP_002766.1 | NKD1 | NP_149110.1 | PLXNA4 | NP_861440.2 |
| ID2 | NP_002157.2 | NOLC1 | NP_001271318.1 | PODXL2 | NP_056535.1 |
| IL33 | NP_001300977.1 | NOTCH1 | NP_060087.3 | RUNX1T1 | NP_001185563.1 |
| KCNK1 | NP_002236.1 | NOTUM | NP_848588.3 | S100A10 | NP_002957.1 |
| LIMCH1 | NP_001317715.1 | NPTX2 | NP_002514.1 | SCRT1 | NP_112599.2 |
| MERTK | NP_006334.2 | NR4A1 | NP_775180.1 | SHTN1 | NP_001245229.1 |
| MGLL | NP_001375244.1 | ODC1 | NP_001274119.1 | SNRPN | NP_001387572.1 |
| MGST1 | NP_665735.1 | OLIG2 | NP_005797.1 | SP8 | NP_945194.1 |
| MLC1 | NP_001363413.1 | PCNA | NP_872590.1 | SP9 | NP_001138722.1 |
| MT3 | NP_005945.1 | PPP1R15A | NP_055145.3 | SRRM4 | NP_919262.2 |
| MYORG | NP_065753.2 | RBM3 | NP_006734.1 | ST18 | NP_055497.1 |
| NAAA | NP_001035861.1 | RRM2 | NP_001025.1 | STMN2 | NP_001186143.1 |
| NIM1K | NP_699192.1 | SERF2 | NP_001186805.1 | STMN3 | NP_056978.2 |
| NKAIN4 | NP_001350676.1 | SIVA1 | NP_068355.1 | STMN4 | NP_001381314.1 |
| NTM | NP_001137531.1 | SLBP | NP_001293003.1 | TIAM2 | NP_001010927.2 |
| NTSR2 | NP_036476.2 | SOCS3 | NP_001365862.1 | TMEFF1 | NP_003683.2 |
| OAF | NP_848602.1 | SUMO2 | NP_001005849.1 | TOX3 | NP_001139660.1 |
| PAQR7 | NP_848509.1 | TEAD2 | NP_001243591.1 | TTC9B | NP_689692.2 |
| PBXIP1 | NP_001304664.1 | TIPIN | NP_001385210.1 | UCHL1 | NP_004172.2 |
| PCDH7 | NP_002580.2 | TMEM132B | NP_001273148.1 | ZNF704 | NP_001028895.1 |
| PLA2G7 | NP_001161829.1 | TNFRSF19 | NP_061117.2 | | |
| PREX2 | NP_079146.2 | TPM4 | NP_003281.1 | | |
| RAMP1 | NP_001295282.1 | UHRF1 | NP_001276980.1 | | |
| RGCC | NP_054778.2 | UNG | NP_550433.1 | | |
| S100A1 | NP_006262.1 | WDR89 | NP_542397.1 | | |
| S1PR1 | NP_001307659.1 | ZFP36 | NP_003398.3 | | |
| SCRG1 | NP_001316526.1 | | | | |
| SDC4 | NP_002990.2 | | | | |
| SERPINE2 | NP_001130002.1 | | | | |
| SFXN5 | NP_001317330.1 | | | | |
| SLC38A3 | NP_006832.1 | | | | |
| SLC39A12 | NP_001269663.1 | | | | |
| SLC4A4 | NP_001128214.1 | | | | |
| SLC6A11 | NP_001304335.1 | | | | |
| SLC7A10 | NP_062823.1 | | | | |
| SLC9A3R1 | NP_004243.1 | | | | |
| SLCO1C1 | NP_001139417.1 | | | | |
| SLITRK2 | NP_001137480.1 | | | | |
| SMPDL3A | NP_001273067.1 | | | | |
| SPARC | NP_001296372.1 | | | | |
| SPARCL1 | NP_001278905.1 | | | | |
| SREBF1 | NP_001308025.1 | | | | |
| TCF7L2 | NP_001336799.1 | | | | |
| THBS4 | NP_001293142.1 | | | | |
| TLCD1 | NP_001153879.1 | | | | |
| TMEM176A | NP_060957.2 | | | | |
| TMEM229A | NP_001129474.1 | | | | |
| TRIL | NP_055632.2 | | | | |
| TST | NP_001257412.1 | | | | |
| TTYH1 | NP_001005367.1 | | | | |
| VCAM1 | NP_001186763.1 | | | | |
| VEGFA | NP_001165101.1 | | | | |

TABLE 2
Preferred bulk lineage biomarkers for quiescent, activation, and differentiation
subpopulations in the astrocyte lineage and in glioma

| Quiescence | Genbank_Acc_Q | Activation | Genbank_Acc_A | Differentiation | Genbank_Acc_D |
|---|---|---|---|---|---|
| ACSS3 | NP_001317171.1 | ALKBH2 | NP_001192108.1 | ACTL6B | NP_057272.1 |
| ADCYAP1R1 | NP_001186566.1 | ANP32B | NP_006392.1 | ADD2 | NP_001171984.1 |
| AGT | NP_001371408.1 | APOD | NP_001638.1 | AFAP1 | NP_001358019.1 |
| AHNAK | NP_076965.2 | ARC | NP_056008.1 | ANK3 | NP_001140.2 |
| ALDH1L1 | NP_001257294.1 | ARL4A | NP_997625.1 | ANKS1B | NP_001190999.1 |
| ALDOC | NP_005156.1 | ATF3 | NP_001193415.1 | ARL4D | NP_001652.2 |
| AQP4 | NP_001304316.1 | BARD1 | NP_001269478.1 | ASPHD1 | NP_859069.2 |
| ARAP2 | NP_056045.2 | BEST3 | NP_001269545.1 | ATL1 | NP_056999.2 |
| ATP1A2 | NP_000693.1 | BTG2 | NP_006754.1 | B4GALNT1 | NP_001263397.1 |
| ATP1B2 | NP_001290192.1 | C11orf24 | NP_001287842.1 | BASP1 | NP_001258535.1 |
| BAALC | NP_001351803.1 | CDCA7L | NP_001120843.1 | BCL11A | NP_001392662.1 |
| BBOX1 | NP_001363190.1 | CENPH | NP_075060.1 | BEND5 | NP_001336724.1 |
| BDH2 | NP_064524.3 | CENPK | NP_001336297.1 | CCNG2 | NP_004345.1 |
| C21orf62 | NP_001155968.2 | CENPU | NP_078905.2 | CCSAP | NP_001397865.1 |
| C3 | NP_000055.2 | CENPW | NP_001273454.1 | CDC42EP3 | NP_001358498.1 |
| CA2 | NP_001280604.1 | CKS2 | NP_001818.1 | CDK5R1 | NP_003876.1 |
| CCDC80 | NP_955806.1 | CLSPN | NP_001177410.1 | CELF3 | NP_001166120.1 |
| CHI3L1 | NP_001267.2 | CSRNP1 | NP_001307489.1 | CELF4 | NP_001340657.1 |
| CLU | NP_001822.3 | DBF4 | NP_001304990.1 | CEP170 | NP_001035864.1 |
| CRB2 | NP_775960.4 | DLL1 | NP_005609.3 | CERS6 | NP_982288.1 |
| CRYAB | NP_001317308.1 | DUSP6 | NP_073143.2 | CHGB | NP_001810.2 |
| CSF1 | NP_757350.2 | EGR1 | NP_001955.1 | CSRNP3 | NP_079245.2 |
| CXCL14 | NP_004878.3 | EGR2 | NP_001397860.1 | CXADR | NP_001193994.1 |
| DHRS3 | NP_001311299.1 | ERF | NP_001299585.1 | DBN1 | NP_001351081.2 |
| DKK3 | NP_001317149.1 | EXOSC9 | NP_001029366.1 | DCX | NP_001356299.1 |
| EDNRB | NP_003982.1 | FBL | NP_001427.2 | DIRAS1 | NP_660156.1 |
| EFEMP1 | NP_001034437.1 | FEN1 | NP_004102.1 | DLX2 | NP_004396.1 |
| EFHD1 | NP_001230181.1 | FOSB | NP_001397998.1 | DPYSL3 | NP_001378.1 |
| ENKUR | NP_001257312.1 | GINS2 | NP_057179.1 | DPYSL5 | NP_001240653.1 |
| F3 | NP_001171567.1 | GPATCH4 | NP_056405.2 | DUSP26 | NP_001292044.1 |
| FADS2 | NP_001268431.1 | HELLS | NP_001276001.1 | DYNC1I1 | NP_001265351.1 |
| FAM107A | NP_001269642.1 | IFRD2 | NP_006755.5 | ELAVL2 | NP_001338390.1 |
| FAM181A | NP_001194000.1 | ITGB3BP | NP_001334077.1 | ELAVL3 | NP_115657.2 |
| FGF1 | NP_149128.1 | JAG1 | NP_000205.1 | ELAVL4 | NP_001311141.1 |
| GFAP | NP_001124491.1 | JUNB | NP_002220.1 | FNBP1L | NP_060207.2 |
| GLIS3 | NP_689842.3 | KLF2 | NP_057354.1 | GAD1 | NP_038473.2 |
| GPR37 | NP_005293.1 | KLF4 | NP_001300981.1 | GDAP1 | NP_001349860.1 |
| HEPACAM | NP_689935.2 | LIMA1 | NP_001107019.1 | GDAP1L1 | NP_001243668.1 |
| HIF3A | NP_690009.1 | LYAR | NP_001139197.1 | GNG2 | NP_001376639.1 |
| HNMT | NP_001019245.1 | MAFF | NP_001155045.1 | GNG3 | NP_036334.1 |
| HOPX | NP_631958.1 | MCM2 | NP_004517.2 | GPR161 | NP_001336564.1 |
| HRH1 | NP_001091683.1 | MCM3 | NP_001257401.2 | HRK | NP_003797.1 |
| HSPB8 | NP_055180.1 | MCM4 | NP_877423.1 | JPH4 | NP_001139500.1 |
| HTRA1 | NP_002766.1 | MCM5 | NP_006730.2 | KALRN | NP_001309927.1 |
| ID3 | NP_002158.3 | MCM6 | NP_005906.2 | KIAA1549 | NP_065961.2 |
| ID4 | NP_001537.1 | METTL1 | NP_075422.3 | KIF5A | NP_001341634.1 |
| IL33 | NP_001300977.1 | MTHFD2 | NP_001397121.1 | KIF5C | NP_004513.1 |
| ITGA6 | NP_001303235.1 | MYADM | NP_001277117.1 | KLF12 | NP_001387070.1 |
| ITM2C | NP_001274169.1 | MYC | NP_001341799.1 | KLHDC8A | NP_001258793.1 |
| ITPKB | NP_001375333.1 | NEU4 | NP_001161073.1 | MAP1B | NP_001311184.1 |
| KCNN3 | NP_001352767.1 | NFKBIZ | NP_001005474.1 | MAPK10 | NP_001338554.1 |
| LAMB2 | NP_002283.3 | NR4A1 | NP_775180.1 | MEX3A | NP_001087194.1 |
| LFNG | NP_001035258.1 | OLIG2 | NP_005797.1 | MLLT11 | NP_006809.1 |
| LIFR | NP_002301.1 | PCNA | NP_872590.1 | MPPED2 | NP_001575.1 |
| LIMCH1 | NP_001317715.1 | PDGFRA | NP_001334756.1 | MTURN | NP_690006.2 |
| LRIG1 | NP_001364273.1 | PDLIM1 | NP_066272.1 | MYT1L | NP_001316778.1 |
| MAOB | NP_000889.3 | PPIH | NP_006338.1 | NOL4 | NP_001371402.1 |
| MGST1 | NP_665735.1 | PPP1R15A | NP_055145.3 | NOVA2 | NP_002507.1 |
| MLC1 | NP_001363413.1 | PTBP1 | NP_114368.1 | NREP | NP_001135948.1 |
| MT3 | NP_005945.1 | PYCR1 | NP_001269210.1 | PAFAH1B3 | NP_001139411.1 |
| NDP | NP_000257.1 | RGS16 | NP_002919.3 | PARP6 | NP_001310450.1 |
| NDRG2 | NP_001269140.1 | RPA2 | NP_001284487.1 | PDZD4 | NP_001290443.1 |
| NMB | NP_066563.2 | SERTAD1 | NP_037508.2 | PKIA | NP_862822.1 |
| NTM | NP_001137531.1 | SLBP | NP_001293003.1 | PLXNA4 | NP_861440.2 |
| NTRK2 | NP_001356481.1 | TIPIN | NP_001385210.1 | POU2F2 | NP_001193955.1 |
| PBXIP1 | NP_001304664.1 | TMEM132B | NP_001273148.1 | PRKCZ | NP_001229803.1 |
| PIFO | NP_001287760.1 | TRIB1 | NP_001269914.1 | PROX1 | NP_002754.2 |
| PLA2G5 | NP_000920.1 | TYMS | NP_001341797.1 | RAB3A | NP_002857.1 |
| PLCD3 | NP_588614.1 | UHRF1 | NP_001276980.1 | RBFOX2 | NP_001076045.1 |
| PLTP | NP_001229849.1 | UNG | NP_550433.1 | REEP1 | NP_001158202.1 |
| PON2 | NP_000296.2 | WEE1 | NP_001137448.1 | RGMB | NP_001353437.1 |
| PROS1 | NP_000304.2 | | | RNF165 | NP_001243687.1 |
| RAMP1 | NP_001295282.1 | | | ROBO2 | NP_001365129.1 |
| RFX4 | NP_001193620.1 | | | RPS6KL1 | NP_001357182.1 |
| RGMA | NP_001159755.1 | | | SBK1 | NP_001019572.1 |
| RHPN1 | NP_443156.2 | | | SCN3A | NP_001075146.1 |
| ROM1 | NP_000318.2 | | | SCN3B | NP_001035241.1 |
| SCARA3 | NP_878185.1 | | | SEZ6 | NP_001277131.1 |
| SDC4 | NP_002990.2 | | | SEZ6L | NP_001171706.1 |
| SFXN5 | NP_001317330.1 | | | SEZ6L2 | NP_001375292.1 |
| SLC1A2 | NP_001182657.1 | | | SH3BP5 | NP_001018009.2 |
| SLC1A3 | NP_001160168.1 | | | SHTN1 | NP_001245229.1 |
| SLC25A18 | NP_001290413.1 | | | SNAP25 | NP_001309831.1 |
| SLC4A4 | NP_001128214.1 | | | SOBP | NP_060483.3 |
| SMOX | NP_787035.1 | | | SRRM3 | NP_001278760.1 |
| SPARC | NP_001296372.1 | | | STMN2 | NP_001186143.1 |
| SPARCL1 | NP_001278905.1 | | | STMN4 | NP_001381314.1 |
| SPOCD1 | NP_001381327.1 | | | STXBP1 | NP_001361243.1 |
| SSPN | NP_001129295.1 | | | SYT1 | NP_001129278.1 |
| TGFB2 | NP_001129071.1 | | | THSD7A | NP_056019.1 |
| TIMP3 | NP_000353.1 | | | TMEM178B | NP_001182207.1 |
| TIMP4 | NP_003247.1 | | | TRIM36 | NP_001017397.1 |
| TMEM176A | NP_060957.2 | | | TTC9B | NP_689692.2 |
| TMEM47 | NP_113630.1 | | | TUBB4A | NP_001276060.1 |
| TRIL | NP_055632.2 | | | UCHL1 | NP_004172.2 |
| TRIM47 | NP_258411.2 | | | WDR47 | NP_001136022.1 |
| TTYH1 | NP_001005367.1 | | | ZBTB8A | NP_001278425.1 |
| TTYH2 | NP_001317382.1 | | | ZNF704 | NP_001028895.1 |
| VAMP5 | NP_006625.1 | | | ZNF711 | NP_001362365.1 |
| VCAM1 | NP_001186763.1 | | | ZNF821 | NP_001363229.1 |

1. A method for treating a glioma in a subject, the method comprising:
(I) determining whether the subject is susceptible to treatment with a wnt modulator by a method comprising allocating cancer cells of the glioma to a multitude of lineage subpopulations; and
(II) treating the glioma in the subject based on the determining in step (I), the treating comprising:
(a) administering a wnt modulator to the subject, and
(b) thereby treating and/or preventing the glioma in the subject.

2. The method of claim 1, wherein said multitude of lineage subpopulations comprises a quiescent subpopulation, an activation subpopulation, and a differentiation subpopulation.

3. The method of claim 1, wherein at least 25% of tumor cells of the glioma are from an activation subpopulation or a differentiation subpopulation and wherein said wnt modulator is an inhibitor of wnt activity (wnt inhibitor).

4. The method of claim 3, wherein said wnt inhibitor is a compound providing a notum polypeptide (Genbank Acc No. NP_848588.3), a porcupine polypeptide (Genbank Acc No. NP_073736.2), secreted frizzled-related protein 1 (sFRP1, Genbank Acc No. NP_003003.3), or is an anti-dickkopf-related protein 1 (DKK1, Genbank Acc No. NP_036374.1) antibody.

5. The method of claim 3, wherein said subject is not planned to undergo chemotherapy.

6. The method of claim 3, wherein said treating comprises administration of said wnt inhibitor until surgical resection of said glioma or a part thereof.

7. The method of claim 1, wherein at least 25% of cancer cells of the glioma are from a quiescent subpopulation and wherein said wnt modulator is an activator of wnt activity (wnt activator).

8. The method of claim 7, wherein said wnt activator is an inhibitor of sFRP1, preferably is WAY-316606 (5-(phenylsulfonyl)-N-4-piperidinyl-2-(trifluoromethyl)-benzenesulfonamide, CAS No. 915759-45-4).

9. The method of claim 7, wherein said treating further comprises administration of chemotherapy and/or radiotherapy.

10. The method of claim 1, wherein said glioma is an astrocytoma, preferably a glioblastoma, more preferably a glioblastoma multiforme.

11. The A method of claim 1, wherein step (a) comprises:
(a) determining at least three lineage biomarkers in a sample of cancer cells of said subject,
(b) comparing said at least three lineage biomarkers determined in step (a) to a lineage reference; and
(c) identifying a subject susceptible to treatment with a wnt modulator based on the result of comparing step (b).

12. The method of claim 11, wherein said method further comprises step (b1) allocating said cancer cells of step (a) to a multitude of lineage subpopulations, preferably wherein said multitude of lineage subpopulations comprises a quiescent subpopulation, an activation subpopulation, and a differentiation subpopulation.

13. A method for evaluating glioma cancer cells comprising
(a) determining at least three lineage biomarkers in a sample of said cancer cells;
(b) comparing said at least three lineage biomarkers determined in step (a) to a lineage reference; and thereby
(c1) evaluating said glioma cancer cells.

14. A database comprising
(I) at least three lineage biomarkers as a glioma cancer cell lineage reference; allocated to
(II) at least one cancer cell subpopulation identifier.

15. (canceled)

16. The method of claim 11, wherein said lineage biomarkers are selected from the list consisting of ALDH1L1, ALDOC, AQP4, ATP1B2, BAALC, CA2, CLU, CXCL14, DHRS3, DKK3, EDNRB, F3, FAM107A, HEPACAM, HOPX, HTRA1, IL33, LIMCH1, MGST1, MLC1, MT3, NTM, PBXIP1, RAMP1, SDC4, SFXN5, SLC4A4, SPARC, SPARCL1, TMEM176A, TRIL, TTYH1, and VCAM1 as biomarkers of a quiescent state, the list consisting of ANP32B, BTG2, DLL1, EGR1, FOSB, HELLS, JUNB, KLF4, LIMA1, LYAR, MCM2, MCM3, MCM5, MCM6, NR4A1, OLIG2, PCNA, PPP1R15A, SLBP, TIPIN, TMEM132B, UHRF1, and UNG as biomarkers of an activation state, and/or the list consisting of ANKS1B, BASP1, CDK5R1, CELF4, DBN1, DCX, DLX2, DPYSL3, ELAVL3, ELAVL4, FNBP1L, GAD1, GNG2, KIF5C, MAP1B, MEX3A, MLLT11, MPPED2, MYT1L, NOL4, NREP, PAFAH1B3, PLXNA4, SHTN1, STMN2, STMN4, TTC9B, UCHL1, and/or ZNF704 as biomarkers of a differentiation state.
